(19) United States
(12) Patent Application Publication
(10) Pub. No.: US 2024/0287498 A1
(43) Pub. Date: Aug. 29, 2024

(54) METHOD FOR ISOLATING NON-VESICULAR MIRNA

(71) Applicant: QIAGEN GmbH, Hilden (DE)

(72) Inventor: Martin SCHLUMPBERGER, Hilden (DE)

(21) Appl. No.: 18/570,344

(22) PCT Filed: Jun. 15, 2022
(86) PCT No.: PCT/EP2022/066288

§ 371 Date: Dec. 14, 2023

(30) Foreign Application Priority Data
Jun. 17, 2021 (EP) 21180184.0
Jun. 24, 2021 (EP) 21181611.1

(51) Int. Cl. C12N 15/10

(52) U.S. Cl. CPC C12N 15/1013

(57) ABSTRACT

The present invention provides methods and kits for isolating cell-free, non-vesicular miRNAs from a biological sample, such as serum and plasma, by using an acidic binding buffer comprising a buffering agent to promote binding of non-vesicular miRNAs to a solid phase comprising anion exchange groups. Furthermore, binding of non-vesicular miRNAs to the solid phase may be improved due to the presence of a crowding agent and by increasing the surface charge density of the solid phase.

### FIELD OF THE INVENTION

The present invention provides improved methods for isolating cell-free, non-vesicular RNA, in particular non-vesicular miRNA, from a biological sample.

### BACKGROUND OF THE INVENTION

Extracellular nucleic acids from cell-free biofluids, such as plasma, serum, or urine, represent important analytes for research and diagnostics. Extracellular nucleic acids include DNA and RNA and are also referred to in the art as cell-free nucleic acids or circulating nucleic acids because such cell-free nucleic acids are often found in body fluids. Of particular relevance are extracellular nucleic acids, such as extracellular RNA (also referred to as “cell-free” or “cfRNA” herein) including miRNA. Extracellular RNA is e.g. found in exosomes and other extracellular vesicles (EVs), which contain mRNA and miRNA besides other RNA transcripts (such as tRNA and Y-RNA). The extracellular RNA comprised in extracellular vesicles is also referred to as vesicular RNA. Hence, in plasma, serum or other cell-free body fluids, miRNA (and other RNAs) is present inside exosomes and other extracellular vesicles (EVs), where it is well protected from ubiquitous, endogenous RNases. Extracellular vesicles may also comprise DNA. However, cell-free RNA such as cell-free miRNA is also present in a separate population, outside of EVs. Cell-free non-vesicular miRNA is often associated with proteins (e.g. miRNAs associated with Ago2 proteins or other Argonaute proteins) and is thereby protected from degradation by RNases. The protection from RNases appears in case of miRNA to be due to tight association with Ago2, and potentially other protective proteins (other Argonaute proteins, miRNA binding proteins such as Nucleophosmin 1, possibly lipoproteins or other protectants). Examples for miRNAs that are found predominantly inside EVs in plasma from healthy donors are let-7a and miR-150, whereas miR-16 and miR-122 are located mostly outside EVs and were shown to be associated with Ago2 protein.

Many methods are known in the prior art in order to isolate extracellular vesicles which in turn allows to recover the vesicular RNA from the isolated EVs. Methods for isolating extracellular vesicles include ultracentrifugation, using e.g. density gradients or sucrose cushions, or size exclusion chromatography or combinations of both (Enderle et al., 2015. Characterization of RNA from exosomes and other extracellular vesicles isolated by a novel spin column-based method. PloS one, 10(8), e0136133). Other methods include polymer-based precipitation, immunocapture (targeting EV membrane proteins) and ultrafiltration. Furthermore, sequential membrane filtration or membrane affinity columns are commonly used for isolation of EVs (e.g. exoRNeasy Kits (QIAGEN, Hilden), see also Srinivasan et al., 2019; Cell, 177(2), 446-462). While anion exchange solid phases (including particles, membranes or modified surfaces) efficiently capture free nucleic acids and EVs (with the contained vesicular nucleic acids), non-vesicular RNA-protein complexes, such as Ago2-miRNA complexes, are typically not bound. This renders the efficient isolation of cell-free, non-vesicular RNA, in particular non-vesicular miRNA, challenging and the prior art methods need improvement.

For the isolation of cell-free non-vesicular miRNA methods are known that are based on immunocapture of Ago2-miRNA complexes. This can be achieved by suitable antibodies, e.g. directly coupled to magnetic beads, or indirectly, using biotinylated antibodies and streptavidin beads. Furthermore, methods for the co-purification of vesicular and non-vesicular miRNA are known, such as the miRNeasy Serum/Plasma Kit (QIAGEN, Hilden). However, these prior art methods are very laborious, limited to very small number of samples at a time, and/or dependent on specific antibodies and therefore expensive. For instance, in case of serum and plasma samples only small volumes can be processed due to the high concentration of other proteins and contaminants present. Moreover, automation of the procedures is in most cases difficult.

Since vesicular and non-vesicular miRNAs are released from cells by different mechanisms (e.g. active secretion of EVs or cell death), there is also a need for providing methods that allow to isolate the vesicular miRNA population and the non-vesicular miRNA population separate from each other. Providing vesicular miRNA and non-vesicular miRNA in separate fractions can provide additional information, including different pathological processes (e.g. toxicity vs. cancer), and may thus provide higher specificity for potential diagnostic applications.

Consequently, there is an urgent need for improved methods for isolating vesicular and non-vesicular RNAs, in particular miRNA, from cell-free or cell-depleted biological samples (such as serum or plasma samples) either simultaneously or as separate fractions. It is thus an object of the present invention to avoid drawbacks of the prior art and to provide such improved methods and kits for performing such methods.

### SUMMARY OF THE INVENTION

The present invention overcomes core drawbacks of the prior art. In particular, the present invention provides improved methods for enriching cell-free, non-vesicular RNA, in particular cell-free non-vesicular miRNA from cell-free or cell-depleted biological samples, in particular cell-free or cell-depleted body fluids such as serum or plasma samples. As is demonstrated by the examples, improved binding of non-vesicular RNA to a solid phase comprising anion exchange groups is achieved by using modified binding conditions that improve the binding of the difficult to capture cell-free non-vesicular miRNA. The modified binding conditions include lowering the pH, increasing the ionic strength of the binding mixture, the addition of molecular crowding agents (e.g. PEG), the use of stronger ion exchange matrices (higher negative charge density), or any combinations of the foregoing. Without wishing to be bound in theory, these modified binding conditions appear to sufficiently weaken the interaction between Ago2 and/or other protective proteins, thereby allowing binding of the vesicular miRNAs to the anion exchange surface, but without degradation of the miRNAs by RNases that might be present in the sample.

Furthermore, by choosing appropriate binding conditions as described herein, it is possible to either isolate vesicular and non-vesicular RNA (such as vesicular miRNA and non-vesicular miRNA) simultaneously in one fraction (to e.g. recover all cell free miRNA), or in separate fractions. The separate enrichment of the vesicular miRNA population and the non-vesicular miRNA population can be achieved by first binding EVs to an anion exchange surface under conditions wherein non-vesicular RNA, such as non-vesicular miRNA is not well-bound and thus remains in the binding mixture. Using modified binding conditions as described herein, the non-vesicular RNA can then be enriched from the remaining sample e.g. by using stronger binding conditions and/or using a different anion exchange matrix that has a higher anionic strength and thus better binding affinity to the non-vesicular RNA, such as non-vesicular miRNA. The term enrichment is used herein in a broad sense and inter alia covers the isolation and purification of the target analyte, i.e. non-vesicular and/or vesicular RNA, such as preferably non-vesicular and/or non-vesicular miRNA.

According to a first aspect, a method is provided for enriching non-vesicular RNA, such as non-vesicular miRNA, from a cell-free or cell-depleted biological sample comprising non-vesicular RNA and optionally extracellular vesicles, wherein the method comprises
(A) preparing a binding mixture comprising
   the biological sample, and
   an acidic binding buffer comprising a buffering agent,
   and binding non-vesicular RNA to a solid phase comprising anion exchange groups;
(B) separating the solid phase with the bound non-vesicular RNA from the binding mixture.

The method according to the first aspect allows to enrich non-vesicular RNA, preferably non-vesicular miRNA, from a cell-free or cell-depleted biological sample comprising non-vesicular RNA and optionally extracellular vesicles. Suitable acidic binding buffers that achieve the efficient capture of the non-vesicular RNA to the solid phase are described herein. Additionally, if extracellular vesicles are comprised in the cell-free or cell-depleted biological sample, also extracellular vesicles (EVs) comprising vesicular RNA, such as vesicular miRNA, can simultaneously bind to the solid phase allowing for simultaneous isolation of non-vesicular and vesicular RNA, such as non-vesicular and vesicular miRNA. The method according to the first aspect can also be used to enrich non-vesicular RNA, such as non-vesicular miRNA, from cell-free or cell-depleted biological samples from which EVs were depleted in advance.

According to a second aspect a method is provided for sequentially enriching extracellular vesicles and non-vesicular RNA, such as non-vesicular miRNA, from a cell-free or cell-depleted biological sample comprising extracellular vesicles and non-vesicular RNA, wherein the method comprises
(X) preparing a binding mixture comprising
   the biological sample, and
   an acidic binding buffer comprising a buffering agent,
   and binding extracellular vesicles to a solid phase comprising anion exchange groups;
   separating the solid phase with the bound extracellular vesicles from the remaining binding mixture, wherein the remaining binding mixture comprises non-vesicular RNA;
(A) preparing a binding mixture comprising
   the remaining binding mixture comprising non-vesicular RNA, and
   optionally an acidic binding buffer comprising a buffering agent,
   and binding non-vesicular RNA to a solid phase comprising anion exchange groups;
(B) separating the solid phase with the bound non-vesicular RNA from the remaining binding mixture of (A);
(C) optionally washing the separated solid phase;
(D) optionally recovering, preferably eluting, non-vesicular RNA from the solid phase.

According to a third aspect, a kit is provided for performing a method according to the first or second aspect, wherein the kit comprises:
(a) a binding buffer according to the present invention;
(b) a solid phase comprising anion exchange groups for binding extracellular RNA and extracellular vesicles;
(c) optionally a washing buffer; and
(d) optionally a lysis buffer.

As disclosed herein, the kit may comprise
(x) an acidic binding buffer comprising a buffering agent, wherein the acidic binding buffer (x) differs from the acidic binding buffer (a), wherein preferably, the acidic binding buffer (a) differs from the acidic binding buffer (x) by one or more of the following features
   (i) it has a lower pH;
   (ii) it has a higher ionic strength; and/or
   (iii) it comprises a crowding agent or comprises a higher concentration of a crowding agent compared to the acidic binding buffer used in step (X); and/or
(b′) a second solid phase comprising anion exchange groups that differs from the solid phase (b), wherein preferably, the solid phase (b) differs from the solid phase (b′) in that (i) it comprises more anion exchange groups; and/or (ii) it comprises stronger anion exchange groups.

Other objects, features, advantages and aspects of the present application will become apparent to those skilled in the art from the following description and appended claims. It should be understood, however, that the following description, appended claims, and specific examples, while indicating preferred embodiments of the application, are given by way of illustration only.

### BRIEF DESCRIPTION OF THE FIGURES

FIG. 1: Ct values of miRNAs recovered using different binding conditions. Vesicular and non-vesicular miRNAs from human plasma samples were enriched by binding to magnetic anion exchange beads carrying histamine functional groups in the presence of different binding buffers (formate, acetate) with varying pH (3, 3.5 or 4) and ionic strength. (A) Ct values of the vesicular miRNA let-7a. (B) Ct values of the non-vesicular miRNA miR-122.

FIG. 2: Ct values of miRNAs recovered using different binding conditions. Vesicular and non-vesicular miRNAs from human plasma samples were enriched using a modified exoRNeasy protocol including binding buffers with different ionic strengths. (A) Ct values of the vesicular miRNA miR-150. (B) Ct values of the non-vesicular miRNA miR-122.

FIG. 3: Ct values of miRNAs recovered using different binding matrices. Recovery of vesicular and non-vesicular miRNAs from magnetic anion exchange beads carrying histamine or His₁₀ peptide as functional groups were compared. For reference, miRNAs were also recovered using the exoRNeasy Maxi Kit. (A) Ct values of the vesicular miRNA let-7a. (B) Ct values of the non-vesicular miRNA miR-122.

FIG. 4: Ct values for miRNAs recovered after binding in presence of PEG or without PEG. Vesicular and non-vesicular miRNAs from human plasma samples were enriched due to binding to magnetic anion exchange beads carrying histamine functional groups in the presence of 5.5% PEG or in the absence of PEG. For reference, miRNAs were also recovered using the exoRNeasy Maxi Kit. (A) Ct values of the vesicular miRNA miR-150. (B) Ct values of the non-vesicular miRNA miR-122.

FIG. 5: Binding of non-vesicular miR-122 from the supernatant obtained from EV binding to magnetic beads or from the flow-through obtained from EV binding to the exoRNeasy spin column. (A) Ct values of the non-vesicular miRNA miR-122. (B) Ct values of the vesicular miRNA miR-150.

### DETAILED DESCRIPTION OF THE INVENTION

As explained in the summary of the invention, the different aspects and embodiments of the invention disclosed herein make important contributions to the art as is also explained in the following.

The term “extracellular vesicle” (EV) or “extracellular vesicles” (EVs) as used herein in particular refers to any type of secreted vesicle of cellular origin. EVs may be broadly classified into exosomes, microvesicles (MVs) and apoptotic bodies. EVs such as exosomes and microvesicles are small vesicles secreted by cells. EVs have been found to circulate through many different body fluids including blood and urine which makes them easily accessible. Due to the resemblance of EVs composition with the parental cell, circulating EVs are a valuable source for biomarkers. Circulating EVs are likely composed of a mixture of exosomes and MVs. They contain nucleic acids, in particular mRNA, miRNA, other small RNAs protected from degradation by a lipid bilayer. The contents are accordingly specifically packaged, and represent mechanisms of local and distant cellular communications. They can transport RNA between cells. EVs such as exosomes are an abundant and diverse source of circulating biomarkers. The cell of origin may be a healthy cell or a cancer cell. The cell of origin may also be an otherwise disease-affected or affected cell, including a stress-affected cell. For instance, the cell may be affected by a neurodegenerative disease. Another example is a stressed cell, such as a cell that underwent ageing. A stressed cell may release more EVs and extracellular DNA. EVs such as exosomes are often actively secreted by cancer cells, especially dividing cancer cells. As part of the tumor microenvironment, EVs such as exosomes seem to play an important role in fibroblast growth, desmoplastic reactions but also initiation of epithelial-mesenchymal transition (EMT) and SC as well as therapy resistance building and initiation of metastases and therapy resistance. There is thus a high interest in analyzing EVs, respectively EV content such as vesicular RNA.

The term “non-vesicular RNA” as used herein refers to RNA comprised in cell-free or cell-depleted biological samples that is not contained in EVs. This cell-free RNA that is not contained in EVs, referred to herein as non-vesicular RNA, is as explained in the background also of particular interest. Typically, such non-vesicular RNA is provided by non-vesicular miRNA which is often associated with proteins (e.g. miRNAs associated with Ago2 proteins or other Argonaute proteins) and is thereby protected from degradation by RNases.

Examples for miRNAs that are found predominantly inside EVs in plasma from healthy donors are let-7a and miR-150, whereas miR-16 and miR-122 are located mostly outside EVs and were shown to be associated with Ago2 protein. These miRNAs can thus be used as markers for vesicular and non-vesicular RNA, respectively.

### The Method According to the First Aspect

According to a first aspect, a method is provided for enriching non-vesicular RNA, such as non-vesicular miRNA, from a cell-free or cell-depleted biological sample comprising non-vesicular RNA and optionally extracellular vesicles, wherein the method comprises
(A) preparing a binding mixture comprising
   the biological sample, and
   an acidic binding buffer comprising a buffering agent,
   and binding non-vesicular RNA to a solid phase comprising anion exchange groups;
(B) separating the solid phase with the bound non-vesicular RNA from the binding mixture.

In embodiments, the method comprises
(C) optionally washing the separated solid phase;
(D) recovering, preferably eluting, non-vesicular RNA from the solid phase.

According to one embodiment, the cell-free or cell-depleted biological sample comprises extracellular vesicles and wherein step (A) comprises binding non-vesicular RNA and extracellular vesicles to the solid phase and step (B) comprises separating the solid phase with the bound non-vesicular RNA and bound extracellular vesicles from the binding mixture.

In embodiments, step (D) comprises recovering from the solid phase non-vesicular RNA and extracellular vesicles or non-vesicular RNA and extracted vesicular RNA.

In preferred embodiments, the non-vesicular RNA to be enriched is non-vesicular miRNA. In one embodiment, vesicular RNA comprised in extracellular vesicles may be enriched together with the non-vesicular RNA.

The binding conditions provided by the present invention enable the effective binding of the difficult to capture non-vesicular RNA, such as non-vesicular miRNA, to a solid phase comprising anion exchange groups. It thereby provides an improved method for the enrichment of cell-free non-vesicular RNA (in particular non-vesicular miRNA) from a cell-free or cell-depleted biological sample. As explained elsewhere herein, the binding of non-vesicular miRNA to an anion exchange surface is especially challenging because proteins, such as Ago2, are associated with the non-vesicular miRNA, which impairs the binding of the cell-free non-vesicular miRNA to the anion exchange groups of the solid phase. The method according to the first aspect overcomes these problems by providing specific binding conditions that weaken the protein-miRNA interaction. This is achieved by the addition of an acidic binding buffer comprising a buffering agent during binding step (A). After binding, the solid phase with the bound non-vesicular RNA is separated from the remaining binding mixture allowing for further processing, e.g. washing and recovering, in particular eluting, the bound non-vesicular RNA and optionally analyzing the eluted non-vesicular RNA by quantitative RT-PCR. Importantly, the binding of extracellular vesicles comprising vesicular RNA, such as vesicular miRNA, to the anion exchange groups is not impaired by the binding conditions used in step (A). Accordingly, if extracellular vesicles (EVs) are additionally comprised in the cell-free or cell-depleted biological sample, they can additionally bind to the anion exchange surface so that after binding, non-vesicular RNA (in particular non-vesicular miRNA) and EVs are bound and thus captured to the anion exchange solid phase. This advantageously allows the simultaneous isolation of non-vesicular and vesicular RNA, such as non-vesicular and vesicular miRNA, from a cell-free or cell-depleted biological sample.

Furthermore, the method according to the first aspect can be used to enrich non-vesicular RNA, preferably non-vesicular miRNA, from a cell-free or cell-depleted biological sample from which extracellular vesicles were depleted. This allows the enrichment of non-vesicular RNA, in particular non-vesicular miRNA, depleted from vesicular RNA. For such depletion of extracellular vesicles, prior art methods may also be used. Furthermore, for the recovery of non-vesicular RNA/miRNA and vesicular RNA/miRNA in separate fractions, the advantageous method according to the second aspect can be used.

The individual steps and preferred embodiments of the method according to the first aspect will now be described in detail.

### Step (A)

Step (A) comprises preparing a binding mixture comprising
the biological sample, and
an acidic binding buffer comprising a buffering agent,
   and binding non-vesicular RNA to a solid phase comprising anion exchange groups. The solid phase comprising anion exchange groups may be comprised in the binding mixture, e.g. in the form of particles, preferably magnetic particles, that provide an anion-exchange surface. The binding mixture may also be passed through a membrane comprising anion exchange groups to thereby bind and thus capture the non-vesicular RNA, such as non-vesicular miRNA, to the membrane. As disclosed herein, EVs, if comprised in the cell-depleted or cell-free biological sample may also bind at the same time to the solid phase. The solid phase comprising anion exchange groups may also be provided by functionalizing at least a portion of the vessel that receives the binding mixture with anion exchange groups. Such anion exchange modified surfaces may also be used as solid phase in the context of the present invention.

As disclosed herein, it is preferred to use anion exchange particles, more preferred magnetic anion exchange particles as solid phase. This simplifies the processing of the particles because they can be processed by the aid of a magnet which is advantageous for automation. Thus, the whole disclosure herein that refers to a solid phase comprising anion exchange groups also specifically applies to the preferred embodiment wherein magnetic anion exchange particles are used as solid phase.

The binding mixture comprises an acidic binding buffer comprising a buffering agent. The acidic binding buffer may be contacted with the biological sample and the solid phase in any order. In embodiments, the acidic binding buffer is first mixed with the biological sample and the obtained mixture is then contacted with the solid phase to prepare the binding mixture according to step (A). As is shown in the examples, the chosen pH and also the used buffering agent influence the extent of non-vesicular miRNA binding to the anion exchange surface of the solid phase. At the same time, binding of EVs (which comprise vesicular miRNA) is not impaired. Therefore, the method according to the first aspect allows adjusting binding conditions that promote binding of non-vesicular RNA and EVs, if EVs are comprised in the cell-free or cell-depleted biological sample which is preferably a body fluid.

### The Acidic Binding Buffer of the Present Invention

### The pH

As disclosed herein and shown in the examples section, the pH of the binding buffer influences whether non-vesicular RNA, in particular non-vesicular miRNA, can be effectively bound to the anion exchange surface or remains unbound in the binding mixture.

In embodiments, the pH of the binding buffer is ≤5.0, preferably ≤4.5 or more preferably ≤4.0. Such an acidic pH ensures efficient binding of the non-vesicular RNA, in particular non-vesicular miRNA, to the anion exchange groups of the solid phase. The acidic pH is chosen so that the target RNA can be bound and may be in the range of 1.5 to 5.0 or 2.0 to 5.0. In embodiments, the pH of the binding buffer is in the range of 2.5 to 5.0, preferably 3.0 to 4.5 or 3.0 to 4.0. As shown by the examples, binding of non-vesicular RNA is improved at a low acidic pH as used in the present method. Without wishing to be bound in theory, it is believed that the low acidic pH used in the present method in particular weakens the miRNA-protein complex sufficiently to allow binding of miRNA to the matrix. Such acidic pH can be determined by the skilled person to ensure good binding.

As is shown in the examples, binding of non-vesicular RNA (in particular non-vesicular miRNA) to the anion exchange groups of the solid phase is more sensitive to pH changes compared to binding of EVs. Thus, while EVs show a similar binding efficiency over a broader range of acidic pH, non-vesicular miRNAs bind less effectively at higher pHs. Advantageously, binding of EVs is not impaired at such lower pH that allows to effectively bind non-vesicular miRNA. This binding behavior can be advantageously used to establish acidic binding conditions in the binding mixture, under which the non-vesicular miRNAs bind with good yield to the anion exchange groups of the solid phase, while binding of EVs to the anion exchange groups of the solid phase is not impaired. This allows for simultaneous binding of non-vesicular and EVs. Furthermore, and as disclosed herein, these findings can be used to establish binding conditions, wherein EVs bind to the anion exchange solid phase, while non-vesicular RNA is bound less effectively and thus remains predominantly in the binding mixture. This differential binding behavior that can be established using the acidic binding conditions disclosed herein can be used in the method according to the second aspect to bind in step (X) EVs to an anion exchange solid phase, while non-vesicular RNA such as non-vesicular miRNA remains in the binding mixture. The non-vesicular RNA can then be recovered from the remaining binding mixture in subsequent step (A) by adjusting the binding mixture so that non-vesicular RNA can be bound (e.g. by lowering the pH, increasing the ionic strength and/or adding a crowding agent) and/or by using in step (A) a stronger anion exchange solid phase. Details are described in conjunction with the method according to the second aspect.

In embodiments, the pH of the binding mixture prepared in step (A) is ≤5.5, ≤5.0 or ≤4.5 or. Preferably, the pH of the binding mixture in step (A) is ≤4. It may be in a range of 2.5 to 5.0, such as 3.0 to 4.5 or 3.0 to 4.0. As disclosed herein, establishing an according acidic pH in the binding mixture allows adjusting the binding conditions and thereby allows to promote the binding of non-vesicular RNA. Binding of EVs is not impaired under these conditions as is demonstrated by the examples. To maintain a stable pH, the pH of the binding mixture prepared in step (A) is preferably within the buffering range of the acidic binding buffer.

Even though it is preferred for many applications that the pH in the binding mixture is established by contacting the biological sample with the acidic binding buffer, the present invention also covers embodiments, wherein the pH value in the binding mixture is adjusted and thus modified after the biological sample was contacted with the binding buffer. Thus, according to one embodiment, the pH value of the binding mixture is adjusted to ensure that the pH of the binding mixture is within the intended range. Suitable pH values are described above and it is referred to the respective disclosure. E.g. the adjustment can be made manually. The pH value of the binding mixture may be determined and then adjusted to the desired pH value by adding appropriate pH modifying substances such as acids or bases. Such procedure can be advantageous if the biological sample has an unusual high or low pH value. However, preferably, the pH of the binding mixture is exclusively established by the addition of the acidic binding buffer according to the invention.

According to an advantageous embodiment, in step (A) the pH of the binding mixture is lower than the pKa of the ionized form of the anion exchange groups of the solid phase. The pH of the binding mixture may be at least 1, at least 1.5, at least 2 or at least 2.5 unit(s) lower than the pKa of the anion exchange group.

As disclosed herein, the binding mixture of step (A) may be prepared by contacting the cell-free or cell-depleted biological sample with the acidic binding buffer and the solid phase comprising anion exchange groups. In embodiments, the binding conditions in the binding mixture of step (A) are exclusively established by contacting the biological sample with the binding buffer and the solid phase. In embodiments, the acidic binding buffer is contacted in step (A) with the biological sample in a ratio of sample to binding buffer that is selected from a range between 10:1 to 1:10, preferably 5:1 to 1:5, 4:1 to 1:4, 3:1 to 1:3, or 2:1 to 1:2, more preferably 1:1. Accordingly, the pH of the binding buffer is chosen with regard to the sample-buffer ratio.

### The Buffering Agent

As disclosed herein, the acidic binding buffer comprises a buffering agent. The buffering agent is chosen such that it has a buffering capacity that includes the desired binding pH value of the binding mixture to allow efficient binding of the non-vesicular RNA, such as in particular the preferred non-vesicular mi-RNA, to the anion exchange surface of the solid phase. In principle any buffer system can be used that is effective within the acidic pH range used.

In embodiments, the acidic binding buffer of step (A) comprises a carboxylic acid based buffering agent. According to one embodiment, the buffering agent comprises a carboxylic acid and a salt of said carboxylic acid. The carboxylic acid may comprise 1 to 3 carboxylic acid groups. In embodiments, the buffering agent comprises a buffer component selected from acetate, formate, citrate, oxalate, propionate, lactate and tartrate.

According to an embodiment illustrated in the examples, the buffering agent comprises a buffer component selected from acetate and formate. As is demonstrated in the examples, acetate and formate are suitable buffering agents and non-vesicular RNA could be effectively captured with good yield when using such buffers at an appropriate pH and/or ionic strength. Binding of EVs to the anion exchange groups of the solid phase is not impaired so that EVs can be co-captured to the anion exchange surface, if EVs are comprised in the cell-free or cell-depleted biological sample which preferably is a body fluid.

In an advantageous embodiment, the acidic binding buffer of step (A) has a pH of ≤4.5 preferably pH≤4 and the binding buffer comprises acetate or formate as buffering agent. As is demonstrated by the examples, acetate and formate were particularly effective in ensuring binding of cell-free non-vesicular miRNA. The pH may be in the range of 2.5 to 4.5, such as 3.0 to 4.0. In a further preferred embodiment, the buffer component is acetate.

The acidic binding buffer may comprise the buffering agent, which preferably is a carboxylic acid based buffering agent as described above, in a concentration of 1M or less, such as 0.75M or less or 0.5M or less. The acidic binding buffer may e.g. comprise the buffering agent in a concentration that lies in the range of 25 mM to 1000 mM, such as 50 mM to 750 mM, 100 mM to 650 mM or 200 mM to 500 mM. The suitable concentration also depends on the volume of the binding buffer that is contacted with the biological sample. As is shown by the examples, e.g. 1 volume of the binding buffer is mixed with 1 volume of the biological sample and the indicated concentrations are particularly effective for such 1:1 ration. Further suitable concentrations for the binding buffer can be easily determined based on this information for other mixing ratios of the cell-free or cell-depleted biological sample and the binding buffer. The resulting binding mixture may then be contacted with the anion exchange solid phase for non-vesicular RNA binding, including non-vesicular RNA and EVs. However, other contacting orders are also feasible and within the scope of the present invention.

According to one embodiment, the binding buffer comprises the buffering agent, which preferably is a carboxylic acid based buffering agent as described above, in a concentration of at least 25 mM, such as at least 50 mM or at least 150 mM, e.g. at least 200 mM. The acidic binding buffer of step (A) may comprise the buffering agent e.g. in a concentration that lies in a range of 25 mM to 650 mM, 50 mM to 600 mM, 150 mM to 550 mM or 200 mM to 500 mM. As is demonstrated in the examples, acidic binding buffers having a pH of ≤4 and comprising the buffering agent, which preferably is a carboxylic acid based buffering agent as described above, in a concentration of ≥200 mM, e.g. in the range of 200 mM to 500 mM, are especially suitable to improve binding of non-vesicular miRNAs to the anion exchange solid support while binding of EVs comprising vesicular miRNAs is not impaired.

As disclosed herein, the binding mixture is prepared by contacting the biological sample with the acidic binding buffer and the binding mixture may include the solid phase comprising anion exchange groups (such as magnetic anion exchange particles). Therefore, the components of the acidic binding buffer which has been described in detail above are also comprised in the binding mixture. It is thus referred to the above disclosure.

As described above, the acidic binding buffer is contacted in step (A) with the biological sample in a ratio of sample to binding buffer that may be selected from a broad range, preferably 4:1 to 1:4, such as 3:1 to 1:3 or 2:1 to 1:2, more preferably 1:1.

In embodiments, the binding mixture of step (A) comprises the buffering agent originating from the binding buffer in a concentration of 500 mM or less, such as 300 mM or less, 250 mM or 200 mM or less. The binding mixture of step (A) may comprise the buffering agent originating from the acidic binding buffer, which preferably is a carboxylic acid based buffering agent as described above, in a concentration that lies in a range of 10 mM to 500 mM, such as 20 mM to 400 mM, 25 mM to 300 mM or 50 mM to 250 mM. In embodiments, the concentration lies in a range of 50 mM to 200 mM.

### Optional Non-Buffering Salt

According to one embodiment, the binding buffer additionally comprises a non-buffering salt. The binding buffer comprises a buffering salt as buffering agent and in addition thereto a non-buffering salt. The non-buffering salt can be used to increase the ionic strength to improve binding of the non-vesicular RNA, such as in particular the non-vesicular miRNA. The salt may be a non-chaotropic salt and preferably is a monovalent salt. Suitable salts include alkali metal salts, such as alkali metal halides. The non-buffering salt may be selected from sodium chloride, potassium chloride, lithium chloride and cesium chloride, preferably selected from sodium chloride and potassium chloride, more preferably the non-buffering salt, if comprised, is sodium chloride. The total salt concentration in the acidic binding buffer used in step (A) is preferably 1M or less, such as 0.75M or less or 0.5M or less. The total salt concentration in the acidic binding buffer may lie in the range of 25 mM to 1000 mM, 50 mM to 750 mM, 100 mM to 650 mM or 200 mM to 500 mM. In the binding mixture of step (A), the total concentration of salt(s) introduced into the binding mixture due to the addition of the binding buffer and optionally further reagent(s) is preferably 500 mM or less, 400 mM or less or 300 mM or less. In embodiments, the total concentration of such introduced salt(s) is 250 mM or less or 200 mM or less.

### The Crowding Agent

According to one embodiment, the binding buffer and/or the binding mixture comprises a crowding agent. As is demonstrated in the examples, addition of a crowding agent (such as PEG) improves binding of non-vesicular RNA, in particular of non-vesicular miRNA, to the anion exchange surface of the solid phase. However, the addition of a crowding agent is not required so that this is an optional, but advantageous component that can be included in the acidic binding buffer and hence the binding mixture of step (A).

Crowding agents are known in the art. Several macromolecules have been described that cause molecular crowding. Examples include, but are not limited to poly(alkylene oxide) polymers (e.g. polyethylene glycol or polypropylene glycol), dextran, synthetic polymers polyvinylpyrrolidone (PVP), or proteins such as serum albumin (e.g. BSA). Preferably, the crowding agent is selected from a poly(alkylene oxide) polymer and dextran.

In an advantageous embodiment, the acidic binding buffer comprises a poly(alkylene oxide) polymers, preferably polyethylene glycol. The comprised polyethylene glycol may have a molecular weight in the range of 5000 Da to 40000 Da, such as 5000 to 30000 Da, 6000 to 20000 Da or 6000 to 10000 Da. In embodiments, the binding buffer comprises polyethylene glycol in a concentration selected from 2% to 30% (w/v), such as 3% to 20% (w/v) or 4% to 15% (w/v). In the examples, a concentration of 5% (w/v) was used.

In embodiments, the concentration of the crowding agent in the binding buffer is in the range of 2% to 10% (w/v), such as 2.5% to 7.5% (w/v) or 4% to 6% (w/v).

The concentration of the crowding agent originating from the binding buffer in the binding mixture of step (A) or added separately to the binding mixture may be in the range of 1% to 15%, such as 1.5%% to 10% or 2% to 5%.

### The Solid Phase Comprising Anion Exchange Groups

As disclosed herein, there are several different options for the solid phase that provides an anion exchange surface due to the presence of anion exchange groups. The solid phase may be provided by particles, membranes/filters or a functionalized surface (e.g. at least a portion of the inner wall of the vessel receiving the sample). The solid phase is not taken into account when determining the concentration of the other components (such as the buffering agent) in the binding mixture.

The solid phase provides an anion exchange surface and thus comprises anion exchange groups at its surface. The solid phase may be provided e.g. by a porous separation means, such as a filter or membrane or may be provided by particles, preferably magnetic particles. The solid phase is preferably provided by particles or a porous membrane or filter. The use of magnetic particles comprising anion exchange surface groups is particularly preferred.

According to an advantageous embodiment, the use of an increased surface charge density or strength by using more or stronger anion exchange groups is especially preferred since increasing the surface charge and/or strength improves non-vesicular RNA binding, in particular non-vesicular miRNA binding, to the solid phase.

Various anion exchange groups comprising functional groups carrying at the binding conditions positive charges may be used that provide the capability to bind negatively charged analytes, such as non-vesicular RNA and EVs. According to one embodiment, the solid phase that is used in step (A) comprises anion exchange groups that comprise at least one primary, secondary, tertiary or quaternary amino group. According to one embodiment, the solid phase is provided by particles, such as magnetic particles, and comprises anion exchange groups that comprise at least one primary, secondary, tertiary or quaternary amino group. According to a further embodiment, the solid phase is provided by a filter or a membrane, e.g. provided in spin columns, and comprises anion exchange groups that comprise at least one primary, secondary, tertiary or quaternary amino group. The amino functionality may also be part of a heterocyclic or heteroaromatic ring, such as the imidazole ring in e.g. histidine or histamine. As disclosed herein, such functional groups may be provided at the surface of the solid phase as monomers, oligomers, or polymers, whereby an increasingly higher density of positive charges on the particle surface is provided. As demonstrated by the examples, non-vesicular miRNAs have a significant higher tendency to bind to anion exchange surfaces having a higher charge density. Therefore, for binding non-vesicular miRNAs, it may be advantageous to use anion exchange groups providing a high charge density.

The anion exchange groups may be coupled as ligands to the surface of the solid phase, such as particles, membranes or other solid phases, as it is well-known in the art.

The surface of the solid phase may comprise anion exchange groups of a single type, however, different types of anion exchange groups may also be used. Suitable anion exchange groups for binding charged molecules such as non-vesicular RNA (in particular non-vesicular miRNA) and EVs are provided by monoamines, diamines, polyamines, and nitrogen-containing aromatic or aliphatic heterocyclic groups. Preferably, the anion exchange group comprises at least one amino group, preferably a primary, secondary or tertiary amino group.

In embodiments, the anion exchange group comprises a group selected from the group consisting of primary, secondary and tertiary amines of the formula
(R)₃N, (R)₂NH, RNH₂ and/or X—(CH₂)ₙ—Y
wherein
X is (R)₂N, RNH or NH₂,
Y is (R)₂N, RNH or NH₂,
R is independently of each other a optionally substituted linear, branched or cyclic alkyl, alkenyl, alkynyl or aryl substituent which may comprise one or more heteroatoms, preferably selected from O, N, S and P, and
n is an integer in the range of from 0 to 20, preferably 0 to 18.

Hence, the anion exchange groups may have an ionisable, in particular protonatable group and optionally may have more than one ionizable group which may be the same or different. A protonatable group preferably is a chemical group which is neutral or uncharged at a high pH value and is protonated at a low pH value, thereby having a positive charge. In particular, the protonatable group is positively charged at the binding pH at which binding of the non-vesicular RNA, in particular non-vesicular miRNA, to the solid phase occurs. In embodiments, the pKa value of the (protonated) protonatable group is in the range of 5 to 13, such as 6 to about 12.5 or 7 to about 12. In embodiments, the pKa value is in the range from 8 to 12 or 9 to 11.5.

Examples of suitable anion exchange groups comprise in particular amino groups such as primary, secondary and tertiary amino groups as well as cyclic amines, aromatic amines and heterocyclic amines. Preferred are tertiary amino groups. The amino groups may bear alkyl, alkenyl, alkynyl and/or aromatic substituents, including cyclic substituents and substituents which together with the nitrogen atom form a heterocyclic or heteroaromatic ring. The substituents may comprise 1 to 20 carbon atoms, such as 1 to 12, 1 to 8, 1 to 6, 1 to 5, 1 to 4, 1 to 3 or 1 or 2 carbon atoms. They may be linear or branched and may comprise heteroatoms such as oxygen, nitrogen, sulfur, silicon and halogen (e.g. fluorine, chlorine, bromine) atoms. In embodiments, the substituents comprise not more than 4, more preferably not more than 3, not more than 2 or not more than 1 heteroatom.

Examples of amine functions are primary amines such as aminomethyl (AM), aminoethyl (AE), aminoalkyl, alkylaminoalkyl, dialkylaminoalkyl such as diethylaminoethyl (DEAE), ethylendiamine, diethylentriamine, triethylentetraamine, tetraethylenpentaamine, pentaethylenhexaamine, trimethylamino (TMA), triethylaminoethyl (TEAE), linear or branched polyethylenimine (PEI), carboxylated or hydroxyalkylated polyethylenimine, jeffamine, spermine, spermidine, 3-(propylamino)propylamine, polyamidoamine (PAMAM) dendrimers, polyallylamine, polyvinylamine, N-morpholinoethyl, polylysine, and tetraazacycloalkanes.

In one embodiment the anion exchange group that is provided as ligand on the surface of the solid phase comprises 1 to 20, 1 to 15 or 1 to 10 ionizable groups, such as the preferred amino groups, per anion exchange group. In preferred embodiments, the anion exchange group of the solid phase that is used for non-vesicular RNA binding comprises 1 to 8, 1 to 6, 1 to 5, 1 to 4, 1 to 3 or 1 to 2 ionizable groups, such as the preferred amino groups, per anion exchange group.

As disclosed therein, the anion exchange groups may comprise at least one amino group that is part of a heterocyclic or heteroaromatic ring. The amino group may be part of an imidazole ring. The anion exchange groups may comprise e.g. histidine and/or histamine. According to one embodiment, the solid phase comprises histamine coupled to a carboxy-modified surface. Alternatively, an imidazole carboxylic acid, such as 4-imidazole acetic acid may be coupled to a surface, such as an amino-modified surface.

According to one embodiment, the anion exchange groups comprise histidine or histamine. The number of histidine groups is preferably at least 3 or at least 4. According to one embodiment, the anion exchange groups are selected from (i) oligo-histidine, wherein the number of histidine monomers is in the range of 4 to 18, such as 5 to 16, 6 to 14, 7 to 13 or preferably 8 to 12, and (ii) a histamine group, optionally wherein the anion exchange groups comprise 1 histamine group per anion exchange group.

According to one embodiment, the anion exchange groups are selected from (i) polyhistidine and (ii) anion exchange groups comprising Bis-Tris groups. According to one embodiment the number of histidine monomers in the polyhistidine is at least 30.

In embodiments, anion exchange particles are used as solid phase for non-vesicular RNA binding in step (A). Magnetic particles are preferred. The magnetic particles may have e.g. ferrimagnetic, ferromagnetic, paramagnetic or superparamagnetic properties. The magnetic properties may be provided to the particles by including into the basic material that forms the particles e.g. iron oxide, e.g. ferrous or ferric oxide or magnetite. Such magnetic particles are well-known in the art and therefore, do not need to be described in detail herein. Anion exchange particles that can be used in the context of the present invention include, but are not limited to, particulate materials that are functionalized with anion exchange groups. As basic material for the particles, any material suitable for anion exchange chromatography may be used, including but not limited to silicon containing materials such as silica and polysilicic acid materials, borosilicates, silicates, anorganic glasses, organic polymers such as poly(meth)acrylates, polyurethanes, polystyrene, agarose, polysaccharides such as cellulose, metal oxides such as aluminum oxide, magnesium oxide, titanium oxide and zirconium oxide, metals such as gold or platinum, sephadex, sepharose, polyacrylamide, divinylbenzene polymers, styrene divinylbenzene polymers, dextrans, and derivatives thereof; glass or silica. In embodiments, the particles are made of or contain a mineral or polymeric material such as silica, glass, quartz, polyethylene, polypropylene, polyvinylidene fluoride, polyacrylonitrile, polyvinylchloride, polyacrylate, methacrylate or methyl methacrylate. Important is that the particles comprise anion exchange groups at their surface and hence provide an anion exchange surface for interaction with the non-vesicular RNA. Such surface can be provided by functionalizing the base material of the particles with suitable anion exchange groups. For functionalizing particles with anion exchange groups in order to provide an anion exchange surface, several methods are feasible and known to the skilled person. The anion exchange groups may be bound directly to the surface of the particles, either covalently or non-covalently, electrostatically and/or may form part of a polymer or other composition which forms a surface coating or which is provided at the surface of the particles. The anion exchange groups may also be precipitated on the particles. According to one embodiment, the anion exchange groups are applied in form of a coating on the particles. A covalent attachment of the anion exchange groups is preferred. The particles may comprise at their surface functionalities for attachment of the anion exchange groups, for example functionalities such as Si—O—Si, Si—OH, (poly-)silicic acid, alcohol, diol or polyol, carboxylate, amine, phosphate or phosphonate. The anion exchange groups may be attached to the solid phase, for example, by using epoxides, (activated) carboxylic acids, silanes, acid anhydrides, acid chlorides, formyl groups, tresyl groups or pentafluorophenyl groups. The functional groups may be attached directly to the solid phase or via (linear or branched) spacer groups, e.g. hydrocarbons such as —(CH₂)n- groups, carbohydrates, polyethylenglycols and polypropylenglycols. In embodiments, the solid phase comprises carboxyl groups for attaching anion exchange groups by covalent attachment using carbodiimide-based reactions, in particular by reacting carboxyl groups of the particles with amino groups comprised in the anion exchange groups. Alternatively, also a polymer composed of monomers comprising the anion exchange group such as an amino functional group can be used as anion exchange material. In certain embodiments, the particles have a silicon containing surface such as a polysilicic acid surface and the anion exchange groups are coupled to said surface by using suitable organosilanes such as an aminosilane.

The anion exchange group may comprise a protonatable group attached to a linker structure. The linker may be a linear, branched or cyclic alkylen, alkenylen or alkynylen group which may comprise 1 to 20 carbon atoms, such as 1 to 12, 1 to 8, 1 to 6, 1 to 5, 1 to 4, 1 to 3 or 1 or 2 carbon atoms. It may further comprise heteroatoms such as oxygen, nitrogen, sulfur, silicon and halogen (e.g. fluorine, chlorine, bromine) atoms, preferably not more than 4, more preferably not more than 3, not more than 2 or not more than 1 heteroatom. In embodiments, the linker group is an alkylene group, in particular a propylene group.

According to one embodiment, the particles comprise a silicon containing surface, preferably a polysilicic acid surface which is derivatized with a silane compound comprising at least one anion exchange group, such as the preferred dialkylaminoalkyl group. Suitable methods involving the use of organosilanes such as aminosilanes are well-known.

In embodiments the anion exchange groups of the solid phase used in step (A) comprise at least one ionizable group, wherein said group is ionizable by protonation, wherein the ionizable group is protonated at the acidic pH of the binding mixture and is neutral or uncharged at a basic pH, such as at a basic pH of at least 8, at least 9 or at least 10. The solid phase used in step (A) may comprise anion exchange groups that have a single positive charge per anion exchange group at the pH of the binding mixture, optionally at a pH of ≤5, such as pH≤4.

As disclosed herein, the anion exchange groups of the solid phase and the binding conditions used in step (A) may be adjusted to establish binding of the non-vesicular RNA to the anion exchange groups of the solid phase, while binding of extracellular vesicles to the anion exchange groups of the solid phase is not impaired under these binding conditions. In embodiments, magnetic anion exchange particles are used, wherein the anion exchange groups of the magnetic particles are provided by an amino group as part of an imidazole ring, preferably wherein the amino group is part of the imidazole ring of histamine, for non-vesicular RNA binding and wherein the acidic binding buffer of step (A) has a pH of ≤5, preferably ≤4. The buffering agent may comprise a buffer component selected from acetate and formate. As disclosed above, the acidic binding buffer may comprise the buffering agent in a concentration that lies in a range 25 mM to 1000 mM, 50 mM to 750 mM, 100 mM to 650 mM or 200 mM to 500 mM and optionally comprises a crowding agent, such as PEG or dextran.

The particles are preferably spherical. The particles may have a mean diameter selected from the ranges of 100 nm to 35 μm, such as 150 nm to 30 μm, 200 nm to 25 μm, 250 nm to 20 μm, 300 nm to 15 μm or 350 nm to 10 μm. Examples include 400 nm to 7.5 μm, 450 nm to 5 μm, 500 nm to 3 μm and 550 nm to 2.5 μm. Suitable exemplary ranges include but are not limited to 100 nm to 10 μm, 150 nm to 7.5 μm, 200 nm to 5 μm, 300 nm to 4 μm, 500 nm to 3.5 μm, 550 nm to 2 μm and 600 nm to 1.5 μm. Particles of the respective sizes and in particular of a smaller size such as 10 μm or less, 7.5 μm or less, preferably 5 μm or less, 2.5 μm or less or 1.5 μm or less are easy to handle and can be well resuspended in the binding mixture. Furthermore, respective small particles provide a large surface area that can bind and accordingly can efficiently collect the non-vesicular RNA from the binding mixture of step (A).

When using particles such as magnetic particles for performing the binding step, the anion exchange particles are not comprised in a column or other device that would prevent the particles from moving in the binding mixture. Instead the particles can move in the binding mixture that is comprised in a container, e.g. when the binding mixture is agitated. Therefore, the particles must be collected from the binding mixture to recover the bound non-vesicular RNA. According to a preferred embodiment, the particles are magnetic. This simplifies the processing of the particles because they can be processed by the aid of a magnet which is advantageous for automation. The particles may have ferrimagnetic, ferromagnetic, paramagnetic or superparamagnetic properties and in embodiments are superparamagnetic. Such properties can be achieved by incorporating a suitable magnetic material into the particles. Suitable methods are known to the skilled person. Preferably, the magnetic material is completely encapsulated e.g. by the silica, polysilicic acid, glass or polymeric material that is used as base material for the particles. In certain preferred embodiments, the nucleic acid binding matrix is a silicon containing particle, preferably a polysilicic acid particle, preferably a magnetic polysilicic acid particle which carries anion exchange groups.

Examples of suitable particles and anion exchange groups are described in WO 2010/072834 A1, DE10 2008 063 001A1, WO2010072821A1, DE 10 2008 063 003, WO 99/29703 and WO0248164 to which it is referred.

The anion exchange particles are added in an amount so that the binding capacity of the anion exchange surface is preferably in excess of the non-vesicular RNA (and optionally EVs) contained in the cell-free or cell-depleted biological sample. This supports a high yield of recovered non-vesicular RNA. Non-limiting examples of suitable amounts of particles (in mg) per ml sample include 0.1 mg to 10 mg, 0.15 mg to 5 mg, 0.2 mg to 3.5 mg, and 0.25 mg to 3 mg. The suitable amount inter alia depends on the sample volume to be processed and the anion exchange particles used and can be determined by the skilled person.

At the end of step (A) non-vesicular RNA, in particular non-vesicular miRNA, is bound to the anion exchange solid phase. Furthermore, if EVs are comprised in the cell-free or cell-depleted biological sample, non-vesicular RNA, in particular non-vesicular miRNA, and EVs are bound to the solid phase.

In an alternative embodiment, at the end of step (A) non-vesicular RNA comprised primarily of non-vesicular RNA, in particular non-vesicular miRNA, is bound to the solid phase. This is achieved by performing an EV depletion step prior to step (A) as is explained in detail elsewhere herein. Such method is also disclosed in conjunction with the method according to the second aspect.

Suitable binding conditions for step (A) are described in the following:
An acidic binding buffer with (i) a pH of 2.5 to 3.5, (ii) acetate as a buffering agent in a concentration of 250 mM to 500 mM, and the use of magnetic anion exchange particles carrying histamine groups;
An acidic binding buffer with (i) a pH of 2.5 to 3.5, (ii) acetate as a buffering agent in a concentration of 250 mM to 500 mM, and the use of magnetic anion exchange particles carrying His10 groups;
An acidic binding buffer with (i) a pH of 2.5 to 3.5, (ii) formate as a buffering agent in a concentration of 200 mM to 500 mM, and the use of magnetic anion exchange particles carrying histamine groups;
An acidic binding buffer with (i) a pH of 2.5 to 3.5, (ii) formate as a buffering agent in a concentration of 200 mM to 500 mM, and the use of magnetic anion exchange particles carrying His10 groups;
An acidic binding buffer with (i) a pH of 2.5 to 3.5, (ii) acetate as a buffering agent in a concentration of 250 mM to 500 mM, (iii) polyethylene glycol as a crowding agent and the use of magnetic anion exchange particles carrying histamine groups;
An acidic binding buffer with (i) a pH of 2.5 to 3.5, (ii) acetate as a buffering agent in a concentration of 250 mM to 500 mM, (iii) polyethylene glycol as a crowding agent and the use of magnetic anion exchange particles carrying His10 groups,
An acidic binding buffer with (i) a pH of 2.5 to 3.5, (ii) formate as a buffering agent in a concentration of 200 mM to 500 mM, (iii) polyethylene glycol as a crowding agent and the use of magnetic anion exchange particles carrying histamine groups;
An acidic binding buffer with (i) a pH of 2.5 to 3.5, (ii) formate as a buffering agent in a concentration of 200 mM to 500 mM, and (iii) polyethylene glycol as a crowding agent and the use of magnetic anion exchange particles carrying His10 groups;
An acidic binding buffer with (i) a pH of ≤4, (ii) acetate as a buffering agent in a concentration of 250 mM to 500 mM, and the use of magnetic anion exchange particles carrying histamine groups;
An acidic binding buffer with (i) a pH of ≤4, (ii) acetate as a buffering agent in a concentration of 250 mM to 500 mM, and the use of magnetic anion exchange particles carrying His10 groups;
An acidic binding buffer with (i) a pH of ≤4, (ii) formate as a buffering agent in a concentration of 200 mM to 500 mM, and the use of magnetic anion exchange particles carrying histamine groups;
An acidic binding buffer with (i) a pH of ≤4, (ii) formate as a buffering agent in a concentration of 200 mM to 500 mM, and the use of magnetic anion exchange particles carrying His10 groups;
An acidic binding buffer with (i) a pH of ≤4, (ii) acetate as a buffering agent in a concentration of 250 mM to 500 mM, (iii) polyethylene glycol as a crowding agent and the use of magnetic anion exchange particles carrying histamine groups;
An acidic binding buffer with (i) a pH of ≤4, (ii) acetate as a buffering agent in a concentration of 250 mM to 500 mM, (iii) polyethylene glycol as a crowding agent and the use of magnetic anion exchange particles carrying His₁₀ groups;
An acidic binding buffer with (i) a pH of ≤4, (ii) formate as a buffering agent in a concentration of 200 mM to 500 mM, (iii) polyethylene glycol as a crowding agent and the use of magnetic anion exchange particles carrying histamine groups;
An acidic binding buffer with (i) a pH of ≤4, (ii) formate as a buffering agent in a concentration of 200 mM to 500 mM, and (iii) polyethylene glycol as a crowding agent and the use of magnetic anion exchange particles carrying His10 groups.

### The Biological Sample

The cell-free or cell depleted biological sample comprises cell-free non-vesicular RNA, such as in particular non-vesicular miRNA, and optionally extracellular vesicles comprising vesicular RNA. According to one embodiment, the cell-free or cell depleted biological sample is or is derived from a body fluid or cell culture liquid. It may be a cell culture supernatant.

In particular, the cell-free or cell depleted biological sample comprising extracellular non-vesicular RNA may be a cell-free or cell-depleted body fluid sample. The cell-free or cell-depleted body fluid sample preferably is or is derived from the following samples by removing cells: whole blood, plasma, serum, lymphatic fluid, urine, liquor, cerebrospinal fluid, synovial fluid, interstitial fluid, ascites, milk, bronchial lavage, saliva, amniotic fluid, semen/seminal fluid, body secretions, nasal secretions, vaginal secretions, wound secretions and excretions. Other body fluids are sweat and tears.

In embodiments, the cell-free or cell depleted biological sample is selected from plasma, serum and cell-free or cell-depleted urine. According to an advantageous embodiment, is selected from plasma and serum. As demonstrated in the examples, the method of the invention is especially suitable for isolating non-vesicular RNA from human plasma samples.

According to one embodiment, prior to step (A) the method comprises removing cells from a body fluid sample, whereby the cell-free or cell depleted biological sample is provided.

In one embodiment, the biological sample comprising non-vesicular RNA, such as in particular non-vesicular miRNA, and optionally extracellular vesicles comprising vesicular RNA is or is derived from a cell culture liquid, in particular a cell culture supernatant. The biological sample may be a sample that was obtained from a cell culture liquid by removing the cells.

Methods to remove cells are known in the art. Common methods to remove cells and provide a cell-free or cell-depleted biological sample include, but are not limited to, centrifugation, filtration and density gradient centrifugation.

According to one embodiment, the biological sample is EV-depleted. Methods for depletion of EVs from a biological sample are described elsewhere herein and it is referred to the respective disclosure. As is known in the art, extracellular vesicles may be isolated and thus depleted from the biological sample by at least one of the following binding to a solid phase, ultracentrifugation, ultrafiltration, gradients, density gradient centrifugation, affinity capture, in particular biochemical affinity capture, antibody capture, size exclusion chromatography or a combination of the foregoing. All these methods may be used in conjunction with the present invention in order to provide an EV-depleted biological sample that can be further processed as described herein in order to enrich and thus isolate non-vesicular RNA. Numerous protocols and commercial products are available for extracellular vesicle/exosome isolation, and are known to the skilled person. E.g. EVs and EV content can be isolated using existing methods, such as exoEasy/exoRNeasy, miRCURY Exosome Isolation kits (or equivalent), ultracentrifugation, size exclusion chromatography, immunocapture, or other methods known in the art. Methods based on the use of volume-excluding polymers, such as PEG, have also been described for the isolation of EVs.

According to another embodiment, the cell-free or cell depleted biological sample comprises cell-free non-vesicular RNA, such as in particular non-vesicular miRNA, and extracellular vesicles (comprising vesicular RNA). As disclosed herein when processing an according cell-free or cell depleted biological sample in step (A), non-vesicular RNA and EVs bind to the solid phase in step (A).

### Step (B)

In step (B) the solid phase with the bound non-vesicular RNA is separated from the remaining binding mixture. Thereby, the solid phase with the bound non-vesicular RNA is collected. For this purpose, any means known in the art can be used. Suitable means include but are not limited to magnetic separation if magnetic particles are used, centrifugation e.g. if non-magnetic particles are used, sedimentation, the application of a vacuum, filtration and the like. When a membrane or other porous material is used as solid phase the processing for separation may be performed e.g. by gravity flow, centrifugation, vacuum or positive pressure. Suitable separation methods are known to the skilled person.

In embodiments, after separating the solid phase with the bound non-vesicular RNA in step (B) the method may comprise
(C) optionally washing the separated solid phase;
(D) recovering, preferably eluting, non-vesicular RNA from the solid phase.

### Wash Step

After separation from the remaining binding mixture, the solid phase with the bound non-vesicular RNA may be washed with a wash buffer. Such a wash step may be performed once or more. As is demonstrated in the examples, a single wash step may be sufficient.

According to one embodiment, in washing step (C) a washing solution. In advantageous embodiments, a washing buffer, is used that has a pH in the range of 3.5 to 6.5, preferably 4.5 to 5.5. According to one embodiment, the pH of the wash buffer is ≤6.5, ≤6 or ≤5.5. Furthermore in embodiments, the pH of the wash buffer is ≥3.5, ≥4 or ≥4.5.

According to one embodiment, the wash solution is a wash buffer that comprises a carboxylic acid based buffering agent, optionally wherein (i) the buffering agent comprises a carboxylic acid and a salt of said carboxylic acid, such as acetate. In embodiments, the wash buffer comprises acetate and has a pH of 5.0.

According to one embodiment, the the wash buffer comprises the buffering agent in a concentration selected from (i) 0.5M or less, 0.4M or less, 0.35M or less, or preferably 0.3M or less; or (ii) in a concentration in the range of 50 mM to 500 mM, such as 100 mM to 400 mM, 150 mM to 350 mM or 200 mM to 300 mM.

### Recovery and Purification of RNA

After separation and preferably washing, the non-vesicular RNA, in particular non-vesicular miRNA, may be recovered, preferably eluted, from the solid phase. Suitable elution protocols for various solid phases, including (magnetic) particles and membranes, are known in the art and the skilled person can choose suitable methods. Suitable recovery and elution protocols are also described in the following.

According to one embodiment, recovering in (D) comprises contacting the solid phase with a lysis buffer, and separating the solid phase from the eluate. The use of a lysis solution for recovery allows to release the bound non-vesicular RNA and furthermore vesicular RNA, if EVs were also bound to the solid phase and separated. As described elsewhere herein, relevant molecular information may be obtained by analyzing RNA molecules present in extracellular vesicles such as exosomes. EVs have been shown to contain various small RNA species, including miRNA, piRNA, tRNA (and fragments thereof), vault RNA, Y RNA, fragments of rRNA, as well as long non-coding RNA, and also mRNA. Accordingly, if EVs are bound in addition to the non-vesicular RNA (which usually is the case if EVs were not depleted from the cell-free or cell-depleted biological sample in advance of step (A)), the recovered RNA comprises non-vesicular and vesicular RNA. The lysis solution may comprise a chaotropic salt, e.g. a guanidinium salt such as guanidinium thiocyanate. The lysis solution may furthermore comprise phenol. In embodiments, the lysis solution used for recovery comprises a chaotropic salt and optionally a detergent. The released RNA may then also be further purified in step (E) from the obtained lysate/eluate, if desired.

The pH of the lysis buffer may be adjusted to pH 7 to 9, preferably 7.5 to 8.5, using guanidinium thiocyanate in a concentration of 1M to and 3M tris(hydroxymethyl)aminomethan (Trizma base) in a concentration of 1M to 3M. Suitable lysis buffers are commercially available (e.g. QIAzol Lysis Reagent, QIAGEN, Hilden) and the pH may be adjusted to pH 7.5 to 8.5. According to one embodiment, the lysis buffer has a pH of 8 and comprises 2M guanidinium thiocyanate and 2M tris(hydroxymethyl)aminomethan (Trizma base). As is demonstrated by the examples, such a lysis buffer is suitable to dissolve nucleoprotein complexes and to lyse bound EVs comprising vesicular RNA. Subsequently, non-vesicular derived miRNAs and vesicular derived miRNAs can be isolated from the obtained eluate by standard methods (e.g. miRNeasy Kits, exoRNeasy Kits, QIAGEN, Hilden).

Non-vesicular RNA may also be eluted by using one or more elution solutions. The non-vesicular RNA may be eluted by using an elution solution that allows for the extracellular RNA to be directly analyzed.

According to one embodiment, elution involves increasing the pH value. Thus, elution can e.g. occur at an elution pH which is higher than the binding pH. E.g. the pH during elution may be increased so that it is above (e.g. ≥1 pH step above) the pKa of the anion exchange matrix. The elution pH is preferably ≥7.0, or ≥7.5. According to one embodiment, the elution solution has a basic pH, preferably of at least 8.0, at least 8.3 or at least 8.5. In embodiments, the pH of the elution solution is ≤9.5 or ≤9.0. According to one embodiment, the elution solution comprises a buffering agent, optionally selected from TRIS, HEPES, HPPS or an ammonia buffer, preferably TRIS.

Furthermore, ionic strength can be used to assist or effect the elution. According to one embodiment, the total salt concentration in the elution solution is at least 500 mM, such as at least 750 mM, at least 1M or at least 1.2M. Such a salt concentration may be advantageous when using an anion exchange solid phase that binds the RNA stronger. For instance, polyethyleneimine-based or poly-histidine-based anion exchange particles, which strongly bind the RNA may require an elution solution comprising such higher salt concentration.

According to one embodiment, the total salt concentration in the elution solution is 500 mM or less, such as 250 mM or less, 200 mM or less, 150 mM or less or 100 mM or less, optionally 50 mM or less. According to a particular embodiment, the elution solution comprises a salt concentration selected from 5 to 250 mM. For instance, the elution solution may comprise 200 mM to 10 mM salt, in particular 200 mM to 10 mM Tris. Such elution solution may have a higher pH as described above to assist the elution.

Elution can also be assisted by heating and/or shaking. A heating step may improve the elution and/or allows using an elution solution that comprises less salt, which can advantageously allow to use the obtained RNA directly for analysis, avoiding subsequent clean-up, e.g. by removal of salts.

As already indicated above, the solid phase comprising the bound non-vesicular RNA and optionally the bound extracellular vesicles may be contacted in in step (D) for recovery with a solution that is an extraction or lysis reagent, optionally wherein the elution solution comprises phenol and/or comprises a chaotropic salt, optionally selected from guanidinium salts, thiocyanate salts, iodide salts, perchlorate salts, trichloroacetate salts and trifluroacetate salts. Optionally such an elution solution has a pH of at least 7.5 or at least 8. This embodiment

Recovery step (D) may also comprise one or more sub-steps. According to one embodiment, the solid phase comprises bound non-vesicular RNA and vesicular RNA and recovery step (D) comprises (aa) lysing the bound extracellular vesicles in the presence of at least one detergent and binding released vesicular RNA to the anion exchange solid phase so that non-vesicular RNA and released vesicular RNA is bound to the solid phase. Advantageously, the detergent that is used for EV lysis does not substantially inhibit binding of the released vesicular RNA to the anion exchange groups of the solid phase. Therefore, the detergent does not need to be removed in order to enable binding of the released vesicular RNA to the anion exchange groups of the solid phase. Step (D) may furthermore comprise (bb) washing the bound released vesicular RNA and non-vesicular RNA. Step (D) may furthermore comprise (cc) eluting the bound released vesicular RNA and non-vesicular RNA from the anion exchange solid phase. Suitable elution solutions were described above and include e.g. an increase in the pH and/or ionic strength to release the bound RNA from the anion exchange solid phase. Sub-step (aa) may comprise contacting the separated anion exchange solid phase comprising the bound non-vesicular RNA and bound extracellular vesicles with an acidic lysis reagent which comprises the at least one detergent suitable to lyse extracellular vesicles so that vesicular RNA is released into the lysate. Detergent-based lysis of extracellular vesicles is also disclosed in the art (see e.g. Osteikoetxea et al Org Biomol Chem 2015 Oct. 14; 13 (38):9775). The concentration of the detergent in the lysis- and rebinding step can be chosen in accordance with the choice of the detergent to achieve efficient lysis of the extracellular vesicles and rebinding. The at least one detergent used in step (aa) for lysing the extracellular vesicles may be selected from a non-ionic surfactant and an anionic detergent. Without being bound to theory, non-ionic surfactants and anionic surfactants are believed to not interfere or not substantially interfere with the binding of the released RNA and the anion exchange particles, when being provided in the lysis mixture. According to a particular embodiment, the detergent used for extracellular vesicle lysis is selected from the group of Triton X-100, sodium dodecyl sulfate, deoxacholate, sarcosyl and/or Ecosurf SA-9. The acidic lysis reagent has an acidic pH that promotes binding of the released vesicular RNA to the anion exchange groups of the solid phase to achieve that non-vesicular RNA and released vesicular RNA is bound to the solid phase. According to a preferred embodiment, the acidic lysis reagent has a pH in the range of 2.5 to 5.5, such as 2.7 to 5.3, 3 to 5 or 3 to 4.7. The pH of the acid lysis reagent may be in the range of 3.5 to 4.5, such as 4.0. It may comprise a buffering agent, such as a carboxylic acid based buffering agent, optionally acetate. The buffering agent may be present in a concentration of ≤500 mM, such as ≤450 mM, ≤400 mM, ≤350 mM, preferably ≤300 mM or ≤250 mM in the acidic lysis reagent. According to one embodiment, the acidic lysis reagent used in step (D) does not comprise a chaotropic salt and/or an organic solvent. According to a preferred embodiment, step (aa) comprises adding a protease. The protease can advantageously assist lysis and therefore, improve the yield of the vesicular RNA and can also support the degradation of any proteins that remain associated with the non-vesicular RNA. Without being bound to theory, a protease, such as proteinase K, is believed to inactivate degradative enzymes that may be present during recovery. Moreover, contaminants may be removed such as protein which bound to the anion exchange groups or solid phase. According to a preferred embodiment, the protease is a proteinase, preferably proteinase K. Such protease digestion step may also be performed at other steps of the present methods, e.g. during binding step (A) and/or during wash step (C). Sub-step (aa) of step (D) may furthermore comprise incubating the lysis mixture to allow lysis of bound extracellular vesicles and direct binding of the released vesicular RNA to the anion exchange groups to provide a solid phase with bound non-vesicular and vesicular RNA. According to a particular embodiment, incubation is performed at room temperature or above. For instance, it may be found suitable to increase the temperature such that an added protease is more active, e.g. by heating to a temperature of 30° C. or more, 35° C. or more, 40° C. or more or 45° C. or more. Suitable temperatures may be readily found in the art by the skilled person. For sub-steps (bb) and (cc) of step (D), washing and elution conditions as described above may be used. Suitable embodiments for lysing EVs and rebinding released vesicular RNA to the anion exchange groups of the solid phase and subsequent wash and elution steps are also disclosed in the PCT application PCT/EP2020/086585 claiming priority to European application 19216752.6 to which we refer.

According to one embodiment, recovering step (D) does not involve lysing bound extracellular vesicles in the presence of at least one detergent. According to one embodiment, recovering step (D) does not involve lysing bound extracellular vesicles in the presence of at least one detergent and binding released nucleic acids, such as preferably RNA to the anion exchange solid phase.

In embodiments, the method comprises
(E) purifying RNA from the eluate.

As disclosed herein, the eluate obtained in recovery step (D) comprises non-vesicular RNA. It may furthermore comprise vesicular RNA if extracellular vesicles also bound in step (A) to the solid phase. In this case, non-vesicular RNA and vesicular RNA is recovered in one fraction. However, if extracellular vesicles are depleted in advance (e.g. using the sequential method according to the present invention) the obtained eluate will predominantly comprise non-vesicular RNA. Either way, the comprised RNA may be further purified using any RNA isolation method. Suitable methods are known in the art and commercially available. Further purifying the obtained RNA can be advantageous for downstream analytical uses. Purification step (E) may be used for further clean-up of the recovered RNA, if necessary, This can be advantageous for downstream analyses that involve methods that require particularly pure RNA input material. However, recovery options are disclosed herein for step (D) that do not require a further clean-up.

A specific recovery and purification protocol is provided by the exoRNeasy Midi/Maxi Kit (QIAGEN, Hilden), which can be incorporated into the methods of the present invention comprising the use of various solid phases, including particles and membranes. After separation of the solid phase with the bound non-vesicular RNA and optional EVs comprising vesicular RNA, a phenol/guanidinium thiocyanate-based lysis buffer (e.g. QIAzol Lysis Reagent, QIAGEN, Hilden) is added to the solid phase. Afterwards, the eluate may be used for further processing, such as RNA purification. In case particles are used as solid phase, an incubation step of about 2 min to 5 min should be performed, optionally by agitating the sample. Subsequently, the supernatant can be collected for further RNA purification. Alternatively, in case a membrane, e.g. in a spin column, is used as solid phase, the tube is centrifuged and subsequently the flow-through is collected for further processing. The sample is agitated, e.g. vortexed, and incubated for about 2 min to 5 min. The lysis buffer dissolves nucleoproteins and lyses the EVs, thereby releasing miRNAs. Next, chloroform is added to the eluate, e.g. supernatant or flow-through, and the sample is agitated, e.g. vortexed. Subsequently, the sample is incubated for about 2 min to 5 min and then centrifuged to remove the phenol. After centrifugation, the sample separates into three phases: an upper, colorless aqueous phase containing RNA; a thin, white interphase; and a lower, red organic phase. The aqueous phase comprising the miRNA originating from non-vesicular RNA and optionally from vesicular RNA is recovered and ethanol is added. The sample is transferred to a spin column (e.g. RNeasy spin column, QIAGEN, Hilden) to bind the RNA. After several washing steps, the RNA can be eluted with RNase-free water.

The obtained and optionally further purified RNA may be analysed using molecular methods. In embodiments, the method of the invention comprises performing a reverse transcription and optionally amplification reaction using the recovered non-vesicular RNA, such as non-vesicular miRNA as template. Furthermore, enriched vesicular RNA (e.g. obtained as one fraction together with the non-vesicular RNA or provided as separate fraction) can be reverse transcribed and optionally amplified for analytical purposes. In embodiments, the reverse transcription and amplification reaction is a quantitative RT-PCR.

For example, the amount of miRNA-16 or miRNA-122 may be quantified to investigate isolation performance of non-vesicular miRNAs. In addition, the amount of let-7a or miRNA-150 may be quantified to investigate isolation performance of vesicular miRNAs.

### Extracellular Vesicle (EV)-Depleted Biological Samples

According to one embodiment, an EV depletion step prior to step (A) is performed to provide a biological sample. Suitable methods for enriching and thus isolating EVs from a cell-free or cell-depleted biological sample are known in the art and are also described elsewhere herein. These methods can be used to provide an extracellular vesicle (EV) depleted cell-free or cell-depleted biological sample that can be used in step (A) to prepare the binding mixture.

In embodiments, the method further comprises prior to preparing in step (A)
(i) contacting the biological sample with a first solid phase, wherein the extracellular vesicles comprising vesicular RNA bind to the first solid phase, wherein the first solid phase is of the same type or of a different type as the solid phase of step (A);
(ii) separating the first solid phase with the bound extracellular vesicles comprising vesicular RNA from the remaining biological sample, wherein the remaining biological sample is an extracellular vesicle-depleted biological sample comprising extracellular non-vesicular RNA.

According to this embodiment, the biological sample is contacted with a first solid phase. In an advantageous embodiment, the first solid phase is of the same type as the solid phase used in step (A).

According to one embodiment, the method further comprises
(iii) recovering, preferably eluting, the extracellular vesicular RNA from the first solid phase.

According to this embodiment, the so depleted and thus recovered EVs are lysed similar to the lysis step as described above in order to release the vesicular RNA, in particular vesicular miRNA, in recovery step (D). Subsequently or in parallel, the extracellular vesicle-depleted biological sample can be further processed according to steps (A) and (B).

### Cell-Depleted Biological Samples

In embodiments, the method comprises removing prior to step (A) cells from a body fluid sample, whereby a cell-depleted or cell-free body fluid sample is provided as biological sample comprising non-vesicular RNA (including non-vesicular RNA and extracellular vesicles comprising vesicular RNA) that is then contacted with the binding buffer and the solid phase in step (B) to prepare the binding mixture. Methods for separating a cell-containing bodily fluid into a, i.e. at least one, cell-containing fraction and a, i.e. at least one, cell-depleted fraction are well-known in the art and therefore, do not need to be described in detail. Common methods include, but are not limited to, centrifugation, filtration and density gradient centrifugation. The different methods may also be combined. The methods are performed so that the integrity of the comprised cells is preserved. This is advantageous because cell breakage during separation would contaminate e.g. the extracellular nucleic acids that are comprised in the cell-depleted fraction with cellular nucleic acids that are released from disrupted cells.

### Automation

The method according to the present invention can be performed manually, or by using automated systems. Automated systems have in particular the advantage that many samples can be processed at the same time and that automated systems are less error prone, because handling errors are avoided. This is a particular advantage where a high number of samples are to be processed, as is the case in many laboratories were samples are analyzed for medical and/or diagnostic purposes. The present method is particularly suitable for automation. Thus, according to one embodiment, the method is performed using an automated system. In this embodiment, it is preferred to use a solid phase selected from magnetic particles, as this simplifies the processing. The magnetic particles including the bound analytes can be processed easily by the aid of a magnetic field, e.g. by using a permanent magnet. This embodiment is e.g. compatible with established robotic systems capable of processing magnetic particles. Here, different robotic systems are used in the art that can be used in conjunction with the present method. As respective systems are well-known in the prior art and are also commercially available (e.g. QIAsymphony®, QIAGEN, Hilden), they do not need any detailed description here. In a further alternative system for processing magnetic particles, the sample comprising the magnetic particles are aspirated into a pipette tip and the magnetic particles are collected in the pipette tip by applying a magnet e.g. to the side of the pipette tip. The remaining sample can then be released from the pipette tip while the collected magnet particles remain due to the magnet in the pipette tip. The collected magnetic particles can then be processed further. Such systems are also well-known in the prior art and are also commercially available (e.g. BioRobot EZ1, QIAGEN, Hilden) and thus, do not need any detailed description here.

### The Method According to the Second Aspect

According to the second aspect, a method is provided for sequentially enriching extracellular vesicles and non-vesicular RNA, such as non-vesicular miRNA, from a cell-free or cell-depleted biological sample comprising extracellular vesicles and non-vesicular RNA, wherein the method comprises
(X) preparing a binding mixture comprising
   the biological sample, and
   an acidic binding buffer comprising a buffering agent,
   and binding extracellular vesicles to a solid phase comprising anion exchange groups;
   separating the solid phase with the bound extracellular vesicles from the remaining binding mixture, wherein the remaining binding mixture comprises non-vesicular RNA;
(A) preparing a binding mixture comprising
   the remaining binding mixture comprising non-vesicular RNA, and
   optionally an acidic binding buffer comprising a buffering agent,
   and binding non-vesicular RNA to a solid phase comprising anion exchange groups;
(B) separating the solid phase with the bound non-vesicular RNA from the remaining binding mixture of (A);
(C) optionally washing the separated solid phase;
(D) optionally recovering, preferably eluting, non-vesicular RNA from the solid phase.

The method according to the second aspect allows for sequential isolation of vesicular and non-vesicular RNA, in particular miRNA, by binding in step (X) extracellular vesicles comprising vesicular RNA to a solid phase and separating the first solid phase with the bound extracellular vesicles from the remaining biological sample (extracellular vesicle (EV)-depleted biological sample comprising non-vesicular RNA). The binding conditions are then modified in step (A). According to one embodiment, the binding conditions are modified by preparing a binding mixture comprising the extracellular vesicle-depleted remaining binding mixture, an anion exchange solid phase and an acidic binding buffer comprising a buffering agent. According to a further embodiment, the binding conditions are modified by using a different anion exchange solid phase which e.g. comprises anion exchange groups which are stronger than the anion exchange groups of the solid phase that was used in step (X) to achieve binding of non-vesicular RNA, such as in particular non-vesicular miRNA, at the so modified binding conditions in step (A). In this embodiment, it is not necessary to add a second acidic binding buffer in step (A) in order to change the chemical composition of the binding mixture compared to step (X). Preparing the binding mixture in step (A) in this case may simply comprise contacting the remaining binding mixture obtained in step (X) with a stronger anion exchange solid phase to achieve binding of the non-vesicular RNA comprised in the binding mixture as is also demonstrated in the examples. Furthermore, these options may also be combined. The modified binding conditions used in step (A) promote binding of non-vesicular RNA to the solid phase comprising anion exchange groups. This can be as explained achieved by using in step (A) a different acidic binding buffer and/or a different anion exchange solid phase than was used in step (X). Afterwards, the solid phase with the bound non-vesicular RNA, in particular non-vesicular miRNA, is separated from the remaining binding mixture. The non-vesicular RNA may then also be recovered from the solid phase as described above in conjunction with the method of the first aspect. Furthermore, the EVs bound in step (A), respectively the contained vesicular RNA may also be recovered from the solid phase using the recovery methods described in conjunction with the first aspect. Preferably, the vesicular and non-vesicular RNA is eluted and thus recovered as separate fractions.

According to one embodiment, in step (X) and step (A) different acidic binding buffers are used. According to one embodiment, the acidic binding buffer used in step (A) differs from the acidic binding buffer used in step (X) by one or more of the following features:
(i) it has a lower pH;
(ii) it has a higher ionic strength;
(iii) it comprises a crowding agent or comprises a higher concentration of a crowding agent compared to the acidic binding buffer used in step (X).

As is demonstrated in the examples, using different binding buffers in step (A) and step (X) provides efficient binding of non-vesicular RNA in step (A) which are not bound or bound to a significant less extent in step (X) (where extracellular vesicles bind).

The individual steps and preferred embodiments of the method according to the second aspect will now be described in detail.

### Step (X)

Step (X) comprises preparing a binding mixture comprising
the cell-depleted or cell-free biological sample, and
an acidic binding buffer comprising a buffering agent, and binding extracellular vesicles to a solid phase comprising anion exchange groups;
   and separating the solid phase with the bound extracellular vesicles from the remaining binding mixture, wherein the remaining binding mixture comprises non-vesicular RNA.

The cell-depleted or cell-free biological sample has been described elsewhere herein in conjunction with the method according to the first aspect and it is referred to the respective disclosure.

The first solid phase that is used in step (X) may be provided by particles, preferably magnetic particles, or by a membrane or other porous support material. Suitable solid phases comprising anion exchange groups such as particles and membranes that allow binding of extracellular vesicles in the presence of an acidic binding buffer are described elsewhere herein in conjunction with the method according to the first aspect and it is referred to the respective disclosure which also applies here. Suitable acidic binding buffers that can be used in step (X) to achieve preferential binding of extracellular vesicles while non-vesicular RNA such as non-vesicular miRNA predominantly remains in the binding mixture and is not bound to the solid phase are described in conjunction with the method according to the first aspect. As explained and demonstrated in the examples, the same types of acidic binding buffers may be used in step (X) and in step (A), e.g. if a weaker anion exchange solid phase is used in step (X). Furthermore, binding of non-vesicular RNA to the anion exchange solid phase is reduced if the pH is less acidic and/or if the ionic strength is lowered. Suitable binding conditions for binding extracellular vesicles to the anion exchange solid phase, while at least a significant portion of non-vesicular RNA remains unbound are also described in the examples and can be determined by the skilled person following the teachings presented herein.

Furthermore, in step (X) the solid phase with the bound extracellular vesicles is separated from the remaining biological sample, wherein the remaining biological sample is an extracellular vesicle-depleted biological sample comprising non-vesicular RNA, in particular non-vesicular miRNA.

Separation procedures are known in the art and discussed elsewhere herein and depend on the type of solid phase used in step (X). The type of the first solid phase may differ from the type of the second solid phase. In preferred embodiments, the type of the first solid phase and the second solid phase are the same.

### Steps (A) and (B)

Steps (A) and (B) of the method according to the second aspect may essentially correspond to steps (A) and (B) of the method according to the first aspect. Therefore, it is referred to the respective disclosure, which also applies with respect to the method according to the second aspect.

Suitable embodiments are described in the following.

The solid phase of step (A) may be of the same or of a different type as the solid phase of step (X). As disclosed herein, using a solid phase with stronger anion exchange groups in step (A) allows binding of non-vesicular RNA to said solid phase without changing the chemical composition of the remaining binding mixture that was obtained in step (X). The remaining binding mixture comprising non-vesicular RNA may simply be contacted with a different solid phase which allows binding of the non-vesicular RNA from the remaining binding mixture. E.g. a stronger anion exchange solid phase may be used for that purpose.

Hence, according to an advantageous embodiment, step (A) comprises using an anion exchange solid phase that differs from the anion exchange solid phase used in step (X) so that binding of non-vesicular RNA, such as non-vesicular miRNA, is improved in step (A) compared to step (X). According to one embodiment, the solid phase used in step (A) differs from the solid phase used in step (X) in that
(i) it comprises more anion exchange groups; and/or
(ii) it comprises stronger anion exchange groups.

As described elsewhere herein, the use of an increased surface charge density or strength of anion exchange groups improves non-vesicular RNA binding to the solid phase in step (A).

As disclosed herein, it is also within the scope of the present invention to change in step (A) the chemical composition of the remaining binding mixture to achieve binding of the non-vesicular RNA. E.g. the remaining binding mixture comprising non-vesicular RNA obtained in step (X) may be modified to prepare the binding mixture of step (A) by one or more of the following to achieve binding of non-vesicular RNA to the anion exchange solid phase in step (A). Such modification may include:
(i) lowering the pH;
(ii) increasing the ionic strength; and/or
(iii) adding a crowding agent.

Such modified binding conditions can be prepared in step (A) by contacting the remaining binding mixture comprising non-vesicular RNA obtained in step (X) with a solution that has (i) a lower acidic pH, (ii) comprises a salt to increase the ionic strength, and/or (iii) comprises a crowding agent, such as PEG. Suitable pH values, salts and crowding agents as well as suitable concentrations for salts and crowding agents were described in detail in conjunction with step (A) of the method according to the first aspect and it is referred to the respective disclosure for the sake of conciseness.

According to one embodiment, preparing the binding mixture in step (A) of the method according to the second aspect comprises contacting the remaining binding mixture comprising non-vesicular RNA obtained from step (X) with an acidic binding buffer that differs from the binding buffer that was used in step (X). As disclosed herein, as anion exchange solid phase, the same type or a different type (e.g. comprising stronger anion exchange groups and/or providing a different format) may be used in step (A) for binding the non-vesicular RNA.

Hence, according to one embodiment, in step (X) and step (A) different acidic binding buffers are used. According to one embodiment, the acidic binding buffer used in step (A) differs from the acidic binding buffer used in step (X) by one or more of the following features:
(i) it has a lower pH;
(ii) it has a higher ionic strength;
(iii) it comprises a crowding agent or comprises a higher concentration of a crowding agent compared to the acidic binding buffer used in step (X).

As is demonstrated in the examples, using different binding buffers in step (A) and step (X) allows efficient binding of non-vesicular RNA in step (A) which are not bound or bound to a significant less extent in step (X) (where extracellular vesicles bind).

In particular, the same acidic binding buffers described above in conjunction with the method according to the first aspect may also be used in step (A) of the method according to the second aspect. It is referred to the respective disclosure which also applies here.

### Further Embodiments

In embodiments, step (X) further comprises
optionally washing the separated solid phase, and
recovering the extracellular vesicles and/or vesicular RNA contained in the extracellular RNA from the separated solid phase.

According to one embodiment, the vesicular RNA of the extracellular vesicles bound to the first solid phase of (X) is recovered. In preferred embodiments, recovering vesicular RNA contained in the separated extracellular vesicles comprises contacting the solid phase with a lysis buffer, optionally wherein the lysis buffer comprises guanidinium thiocyanate, and separating the solid phase from the eluate that comprises vesicular RNA. Details of this recovery step were already described in conjunction with step (D) above and we refer to the respective disclosure. The same recovery step can be used in order to recover the vesicular RNA that is contained in the extracellular vesicles and that were bound to and separated with the solid phase in step (X).

Recovering the extracellular vesicles and/or vesicular RNA contained in the extracellular RNA may be performed in any order or in parallel to (D) optionally recovering, preferably eluting, non-vesicular RNA from the solid phase.

According to one embodiment, the same type of solid phase is used in step (X) and step (A). Advantageously, the same solid phase comprising anion exchange groups can be used in step (X) and step (A). The preferential binding conditions for extracellular vesicles in step (X) can be established by adjusting the binding conditions in step (X) so that extracellular vesicles bind to the solid phase in step (X) while non-vesicular RNA at least partially remains in the binding mixture and is not captured to the solid phase in step (X). Preferably, the binding conditions are chosen such in step (X) that non-vesicular RNA is predominantly not bound in step (X) and thus can be recovered from the remaining binding mixture in step (A). Suitable binding conditions are disclosed herein and illustrated in the examples. Preferably, the solid phase is provided by particles, preferably magnetic particles. Suitable embodiments are described above in conjunction with the method according to the first aspect. Using the same solid phase, such as magnetic particles comprising anion exchange groups, in step (X) and step (A) allows to provide a single vessel for the magnetic particles that can be used in step (X) and step (A). This is advantageous as it facilitates automation. In a different embodiment, a different solid phase is used in step (X) and step (A). In this case, it is preferred that the solid phase used in step (A) differs from the solid phase used in step (X) in that (i) it comprises more anion exchange groups; and/or (ii) it comprises stronger anion exchange groups. E.g. quaternary anion exchange groups provide a very strong binding affinity for non-vesicular RNA, such as non-vesicular miRNA. Further suitable examples are described elsewhere herein and in particular in conjunction with the method according to the first aspect. Furthermore, different forms of solid phases may be used in steps (X) and step (A), such as magnetic particles in one binding step and a membrane in the other binding step.

Furthermore, a solid phase comprising anion exchange groups may also be provided by functionalizing with anion exchange groups at least a portion of the vessel that receives the biological sample/binding mixture in step (A). Such solid phase may also be used in step (X).

Subsequently, the RNA, preferably miRNA, may be isolated from each of the fractions (vesicular miRNA and non-vesicular miRNA) using commercially available methods as described for method of the first aspect above (e.g. miRNeasy Kits, QIAGEN, Hilden). The isolated vesicular RNA and non-vesicular RNA can then each be analyzed, e.g. by quantitative RT-PCR.

### The Kit According to the Third Aspect

According to a third aspect, a kit is provided for performing the method according to the first and second aspects, comprising
(a) an acidic binding buffer comprising a buffering agent;
(b) a solid phase comprising anion exchange groups suitable for binding non-vesicular RNA under the conditions established by the acidic binding buffer (a);
(c) optionally a wash solution; and
(d) optionally a recovery solution.

Advantageously, the kit can be provided with different buffer sets and/or different solid phases comprising anion exchange groups that allow the user to choose between the enrichment of non-vesicular RNA as separate fraction or the enrichment of all extracellular RNA comprising non-vesicular and vesicular RNA. Furthermore, as disclosed herein, extracellular vesicles or vesicular RNA can be also be enriched as separate fraction. The kits according to the invention thereby provide a great flexibility for the user.

In embodiments, the kit comprises
(x) an acidic binding buffer comprising a buffering agent, wherein the acidic binding buffer (x) differs from the acidic binding buffer (a), wherein preferably, the acidic binding buffer (a) differs from the acidic binding buffer (x) by one or more of the following features
   (i) it has a lower pH;
   (ii) it has a higher ionic strength; and/or
   (iii) it comprises a crowding agent or comprises a higher concentration of a crowding agent compared to the acidic binding buffer used in step (X).

The advantages of using different binding buffers in steps (X) and (A) has been described elsewhere herein and it is referred to the respective disclosure,

According to one embodiment, the kit comprises
(b′) a second solid phase comprising anion exchange groups that differs from the solid phase (b), wherein preferably, the solid phase (b) differs from the solid phase (b′) in that (i) it comprises more anion exchange groups; and/or (ii) it comprises stronger anion exchange groups.

The advantages of using different solid phases in steps (X) and (A) has been described elsewhere herein and it is referred to the respective disclosure,

According to one embodiment, the kit comprises a binding buffer (x) and a second solid phase (b′) and wherein the second solid phase (b′) is suitable for binding extracellular vesicles under the conditions established by the acidic binding buffer (x).

As described herein, the use of a binding buffer (x) in combination with a solid phase (b′) allows to establish binding conditions that allows to efficiently and preferentially capture extracellular vesicles to the solid phase, while non-vesicular RNA is not or less well bound and thus remains in the binding mixture. The non-vesicular RNA can then be recovered from the remaining binding mixture that was depleted from extracellular vesicles using the binding buffer (a) and the solid phase (b). Furthermore, as disclosed herein, it is also possible to use the same solid phase (b) in the kit and only provide different binding buffers (x) and (a) to establish the different binding conditions that allow the sequential isolation of extracellular vesicles and non-vesicular RNA. This advantageously allows to recover vesicular RNA and non-vesicular RNA in different fractions for the subsequent analysis.

According to one embodiment, the kit has one or more of the following characteristics:
(i) the acidic binding buffer (a) has one or more of the characteristics as defined according to the present invention,
(ii) the solid phase (b) has one or more of the characteristics as defined according to the present invention, wherein preferably the solid phase is provided by magnetic particles;
(iii) the wash buffer (c) has one or more of the characteristics as defined according to the present invention; and/or
(iv) the recovery solution (d) is a lysis solution comprising guanidinium thiocyanate.

Furthermore, the kit may comprise a recovery/elution solution as described in conjunction with the method according to the first aspect.

### Use According to the Fourth Aspect

According to a fourth aspect, the use of an acidic binding buffer comprising a buffering agent for isolating extracellular RNA comprised in a biological sample is provided, wherein the extracellular RNA binds to a solid phase comprising anion exchange groups in the presence of the buffering agent. The binding buffer may be as defined in conjunction with the method of the first aspect and it is referred to the respective disclosure.

According to one embodiment, the use comprises performing the method according to the first or second aspects. According to one embodiment, the use comprises sequentially isolating (i) extracellular vesicular miRNA and (ii) extracellular non-vesicular miRNA. According to an alternative embodiment, the use comprises simultaneously isolating (i) extracellular vesicular miRNA and (ii) extracellular non-vesicular miRNA.

### Further Embodiments

Embodiments of the present invention are described again and in further detail in the following. The present invention in particular discloses and provides the following items:
1. A method for enriching non-vesicular RNA, such as non-vesicular miRNA, from a cell-free or cell-depleted biological sample comprising non-vesicular RNA and optionally extracellular vesicles, wherein the method comprises
(A) preparing a binding mixture comprising
   the biological sample,
   an acidic binding buffer comprising a buffering agent, and binding non-vesicular RNA to a solid phase comprising anion exchange groups;
(B) separating the solid phase with the bound non-vesicular RNA from the binding mixture.
2. The method according to item 1, wherein the method comprises
(C) optionally washing the separated solid phase;
(D) recovering, preferably eluting, non-vesicular RNA from the solid phase.
3. The method according to item 1 or 2, wherein the cell-free or cell-depleted biological sample comprises extracellular vesicles and wherein step (A) comprises binding non-vesicular RNA and extracellular vesicles to the solid phase and step (B) comprises separating the solid phase with the bound non-vesicular RNA and bound extracellular vesicles from the binding mixture.
4. The method according to item 2 and 3, wherein step (D) comprises recovering from the solid phase non-vesicular RNA and extracellular vesicles or non-vesicular RNA and extracted vesicular RNA.
5. The method according to item 1 or 2, wherein the cell-free or cell-depleted biological sample is depleted of extracellular vesicles.
6. A method for sequentially enriching extracellular vesicles and non-vesicular RNA, such as non-vesicular miRNA, from a cell-free or cell-depleted biological sample comprising extracellular vesicles and non-vesicular RNA, wherein the method comprises
(X) preparing a binding mixture comprising
   the biological sample,
   an acidic binding buffer comprising a buffering agent,
   and binding extracellular vesicles to a solid phase comprising anion exchange groups;
   separating the solid phase with the bound extracellular vesicles from the remaining binding mixture, wherein the remaining binding mixture comprises non-vesicular RNA;
(A) preparing a binding mixture comprising
   the remaining binding mixture comprising non-vesicular RNA,
   optionally an acidic binding buffer comprising a buffering agent,
   and binding non-vesicular RNA to a solid phase comprising anion exchange groups;
(B) separating the solid phase with the bound non-vesicular RNA from the remaining binding mixture of (A);
(C) optionally washing the separated solid phase;
(D) optionally recovering, preferably eluting, non-vesicular RNA from the solid phase.
7. The method according to item 6, wherein preparing a binding mixture in step (A) comprises contacting the remaining binding mixture comprising non-vesicular RNA obtained in step (X) with a different anion exchange solid phase than the solid phase that was used in step (X).
8. The method according to item 6 or 7, wherein preparing a binding mixture in step (A) comprises modifying the remaining binding mixture comprising non-vesicular RNA obtained in step (X) to achieve binding of non-vesicular RNA to the anion exchange solid phase used in step (A), wherein modifying includes one or more of
(i) lowering the pH,
(ii) increasing the ionic strength, and/or
(iii) adding a crowding agent, such as polyethylene glycol.
9. The method according to item 7, wherein the chemical composition of the binding mixture is not changed compared to step (X) and wherein binding of the non-vesicular RNA is achieved by the use of a different anion exchange solid phase.
10. The method according to any one of items 6 to 8, wherein step (A) comprises contacting the remaining binding mixture comprising non-vesicular RNA obtained from step (X) with an acidic binding buffer comprising a buffering agent to prepare the binding mixture, wherein in step (X) and step (A) different acidic binding buffers are used.
11. The method according to any one of items 6 to 8 or 10, wherein step (A) comprises contacting the remaining binding mixture comprising non-vesicular RNA obtained in step (X) with a different anion exchange solid phase than the solid phase used in step (X) and with an acidic binding buffer comprising a buffering agent to prepare the binding mixture, wherein in step (X) and step (A) different acidic binding buffers are used.
12. The method according to items 10 or 11, wherein the acidic binding buffer used in step
(A) differs from the acidic binding buffer used in step (X) by one or more of the following features:
(i) it has a lower pH;
(ii) it has a higher ionic strength; and/or
(iii) it comprises a crowding agent or comprises a higher concentration of a crowding agent compared to the acidic binding buffer used in step (X).
13. The method according to any one of items 6 to 12, wherein step (A) comprises using an anion exchange solid phase that differs from the anion exchange solid phase used in step (X) so that binding of non-vesicular RNA, such as non-vesicular miRNA, is improved in step (A) compared to step (X).
14. The method according to any one of items 6 to 13, wherein the solid phase used in step (A) differs from the solid phase used in step (X) in that
(i) it comprises more anion exchange groups; and/or
(ii) it comprises stronger anion exchange groups.
15. The method according to any one of items 1 to 14, wherein the pH of the acidic binding buffer of step (A) and optionally step (X) is ≤5.0, preferably ≤4.5 or ≤4.0.
16. The method according to item 15, wherein the pH of the acidic binding buffer of step (A) and optionally step (X) is in the range of 2.5 to 5.0, preferably 3.0 to 4.5 or 3.0 to 4.0.
17. The method according to any one of items 1 to 16, wherein the acidic binding buffer of step (A) and optionally step (X) comprises a carboxylic acid based buffering agent.
18. The method according to item 17, wherein the buffering agent comprises a carboxylic acid and a salt of said carboxylic acid.
19. The method of item 18, wherein the carboxylic acid
(i) comprises 1 to 3 carboxylic acid groups;
(ii) is aliphatic; and/or
(iii) is saturated,
optionally wherein the buffering agent comprises a buffer component selected from citrate, oxalate, formate, acetate, propionate, lactate and tartrate.
20. The method according to any one of items 17 to 19, wherein the buffering agent comprises a buffer component selected from acetate and formate.
21. The method according to any one of items 1 to 20, (i) wherein the acidic binding buffer of step (A) and optionally step (X) comprises the buffering agent in a concentration of 1M or less, such as 0.75M or less or 0.5M or less; or (ii) wherein the acidic binding buffer of step (A) and optionally step (X) comprises the buffering agent in a concentration that lies in the range of 25 mM to 1000 mM, 50 mM to 750 mM, 100 mM to 650 mM or 200 mM to 500 mM.
22. The method according to any one of items 1 to 21, wherein the acidic binding buffer of step (A) and optionally step (X) comprises a non-buffering salt.
23. The method according to any one of items 1 to 22, wherein the acidic binding buffer and/or the binding mixture of step (A) and optionally step (X) comprises a crowding agent.
24. The method according to item 23, wherein the crowding agent is selected from a poly(alkylene oxide) polymer and dextran.
25. The method according to item 24, wherein the crowding agent is polyethylene glycol, wherein preferably, the polyethylene glycol has a molecular weight in the range of 5000 Da to 40000 Da, such as 5000 Da to 30000 Da, 6000 Da to 20000 Da or 6000 Da to 10000 Da.
26. The method according to any one of items 1 to 25, wherein the acidic binding buffer in (A) is selected from the following:
(aa) it comprises (i) a pH of 2.5 to 5, preferably 3 to 4, and (ii) acetate as a buffering agent in a concentration of 250 mM to 500 mM;
(bb) it comprises (i) a pH of 2.5 to 5, preferably 3 to 4, and (ii) formate as a buffering agent in a concentration of 200 mM to 500 mM;
(cc) it comprises (i) a pH of 2.5 to 5, preferably 3 to 4, (ii) acetate as a buffering agent in a concentration of 250 mM to 500 mM, and (iii) a crowding agent, preferably polyethylene glycol;
(dd) it comprises (i) a pH of 2.5 to 5, preferably 3 to 4, (ii) formate as a buffering agent in a concentration of 250 mM to 500 mM, and (iii) a crowding agent, preferably polyethylene glycol;
(ee) it comprises (i) a pH of ≤5, preferably ≤4, and (ii) acetate as a buffering agent in a concentration of 250 mM to 500 mM;
(ff) it comprises (i) a pH of ≤5 and (ii) formate as a buffering agent in a concentration of 200 mM to 500 mM;
(gg) it comprises (i) a pH of ≤5, (ii) acetate as a buffering agent in a concentration of 250 mM to 500 mM and (iii) a crowding agent, preferably polyethylene glycol; and
(hh) it comprises (i) a pH of ≤5, (ii) formate as a buffering agent in a concentration of 250 mM to 500 mM and (iii) a crowding agent, preferably polyethylene glycol.
27. The method according to any one of items 1 to 26, wherein the solid phase that is used in step (A) is provided by particles, preferably magnetic particles or a porous membrane or filter.
28. The method according to any one of item 27, wherein the solid phase that is used in step (A) comprises anion exchange groups that comprise at least one primary, secondary, tertiary or quaternary amino group.
29. The method according to item 28, wherein the anion exchange group of the solid phase comprises at least one amino group, wherein the amino group is part of an imidazole ring, preferably wherein the amino group is part of the imidazole ring of histamine.
30. The method according to any one of items 1 to 29, wherein in (A) the binding buffer, having an acidic pH, comprising a buffering agent and optionally comprising a crowding agent, and the solid phase are selected from the following:
(aa) (i) a pH of 2.5 to 5, preferably 3 to 4, (ii) acetate as a buffering agent in a concentration of 250 mM to 500 mM, and (iii) magnetic anion exchange particles carrying histamine and/or His 10;
(bb) (i) a pH of ≤5, preferably ≤4, (ii) acetate as a buffering agent in a concentration of 250 mM to 500 mM, and (iii) magnetic anion exchange particles carrying histamine and/or His₁₀.
31. The method according to any one of items 1 to 30, wherein the cell-free or cell depleted biological sample has one or more of the following characteristics:
(i) it is or is derived from a body fluid or cell culture liquid;
(ii) it is or is derived from the following samples by removing cells: whole blood, plasma, serum, lymphatic fluid, urine, liquor, cerebrospinal fluid, synovial fluid, interstitial fluid, ascites, milk, bronchial lavage, saliva, amniotic fluid, semen/seminal fluid, body secretions, nasal secretions, vaginal secretions, wound secretions and excretions;
(iii) it is selected from plasma, serum and cell-free or cell-depleted urine;
(iv) it is selected from plasma and serum,
optionally wherein prior to step (A) the method comprises removing cells from a body fluid sample, whereby the cell-free or cell depleted biological sample is provided.
32. The method according to any one of items 2 to 31, wherein washing step (C) comprises using a washing solution, preferably a washing buffer, that has a pH in the range of 3.5 to 6.5, preferably 4.5 to 5.5.
33. The method according to item 32, wherein the wash solution is a washing buffer that comprises a carboxylic acid based buffering agent, optionally wherein (i) the buffering agent comprises a carboxylic acid and a salt of said carboxylic acid, such as acetate.
34. The method according to item 32 or 33, wherein the wash buffer comprises the buffering agent in a concentration selected from (i) 0.5M or less, 0.4M or less, 0.35M or less, or preferably 0.3M or less; or (ii) in a concentration in the range of 50 mM to 500 mM, such as 100 mM to 400 mM, 150 mM to 350 mM or 200 mM to 300 mM.
35. The method according to any one of items 2 to 34, wherein recovering in (D) comprises contacting the solid phase with a lysis buffer, optionally wherein the lysis buffer comprises guanidinium thiocyanate, and separating the solid phase from the eluate.
36. The method according to any one of items 2 to 34, wherein recovering in (D) comprises contacting the solid phase with an elution solution and separating the solid phase from the eluate, wherein the elution solution has one or more of the following characteristics:
(i) it has higher pH than the pH of the binding mixture of step (A), wherein preferably the pH of the elution solution is at least 7.0 or at least 7.5, such as at least 8.0, at least 8.3 or at least 8.5;
(ii) it has a pH that it is above the pKa of the ionized form of the anion exchange groups of the solid phase, wherein preferably the pH of the elution solution is at least 1 unit above the pKa of the anion exchange groups;
(iii) it has a basic pH, wherein the pH of the elution solution is 9.5 or less or 9.0 or less;
(iv) it comprises a buffering agent, wherein optionally the buffering agent is selected from TRIS, HEPES, HPPS, and an ammonia buffer, preferably the buffering agent is TRIS
(v) it has a total salt concentration of at least 500 mM, such as at least 750 mM, at least 1M or at least 1.2M;
(vi) it has a total salt concentration of 500 mM or less, such as 250 mM or less, 200 mM or less, 150 mM or less or 100 mM or less, optionally 50 mM or less;
(vii) it has a salt concentration selected from 5 mM to 200 mM, such as 10 mM to 200 mM, preferably the elution solution comprises TRIS at a concentration of 10 mM to 200 mM; and/or
(viii) the elution solution is 10 mM Tris, pH 8.5.
37. The method according to any one of items 2 to 36, wherein the method comprises
(E) purifying RNA from the eluate.
38. The method according to any one of item 6 to 37 when depending on item 6, wherein step (X) further comprises
   optionally washing the separated solid phase, and
   recovering the extracellular vesicles and/or vesicular RNA contained in the extracellular RNA from the separated solid phase.
39. The method according to item 38, wherein recovering vesicular RNA contained in the separated extracellular vesicles comprises contacting the solid phase with a lysis buffer, optionally wherein the lysis buffer comprises guanidinium thiocyanate, and separating the solid phase from the eluate that comprises vesicular RNA.
40. The method according to any one of items 6 to 39 when depending on item 6, wherein the same type of solid phase is used in step (X) and step (A).
41. The method according to any one of items 1 to 40, wherein the non-vesicular RNA comprises or predominantly consists of non-vesicular miRNA.
42. The method according to any one of items 1 to 41, wherein the vesicular RNA comprises vesicular miRNA.
43. The method according to item 1 and the dependent items when depending on item 1, wherein the recovered RNA comprises non-vesicular miRNA and vesicular miRNA.
44. The method according to item 6 and the dependent items when depending on item 6, wherein vesicular miRNA and non-vesicular miRNA are provided as separate fractions.
45. The method according to any one of items 1 to 44, wherein the non-vesicular RNA to be enriched is non-vesicular miRNA, wherein the method comprises
(A) preparing a binding mixture comprising
   the biological sample,
   wherein the biological sample is selected from plasma and serum and optionally is extracellular vesicle-depleted,
   an acidic binding buffer comprising a buffering agent,
   wherein the pH of the binding buffer is ≤5.0, preferably ≤4.5 or ≤4.0,
   wherein the buffering agent comprises a carboxylic acid based buffer component, preferably selected from acetate and formate,
   (i) wherein the acidic binding buffer comprises the buffering agent in a concentration of 1M or less, such as 0.75M or less or 0.5M or less; or (ii) wherein the acidic binding buffer comprises the buffering agent in a concentration that lies in the range of 25 mM to 1000 mM, 50 mM to 750 mM, 100 mM to 650 mM or 200 mM to 500 mM,
   and binding non-vesicular RNA to a solid phase comprising anion exchange groups,
   wherein the solid phase that is used in step (A) is provided by particles, preferably magnetic particles or a membrane,
   wherein the anion exchange group of the solid phase comprises at least one amino group, wherein the amino group is part of an imidazole ring, preferably wherein the amino group is part of the imidazole ring of histamine;
(B) separating the solid phase with the bound non-vesicular RNA from the remaining binding mixture;
(C) optionally washing the separated solid phase;
(D) recovering, preferably eluting, non-vesicular RNA from the solid phase, optionally wherein recovering in (D) comprises contacting the solid phase with a lysis buffer, optionally wherein the lysis buffer comprises guanidinium thiocyanate, and separating the solid phase from the eluate; and
(E) optionally purifying RNA from the eluate.
46. The method according to any one of items 6 to 43, wherein the non-vesicular RNA to be enriched is non-vesicular miRNA and the extracellular vesicles comprise vesicular miRNA, wherein the method comprises
(X) preparing a binding mixture comprising
   the biological sample,
   an acidic binding buffer comprising a buffering agent, and binding extracellular vesicles to a solid phase comprising anion exchange groups;
   separating the solid phase with the bound extracellular vesicles from the remaining binding mixture, wherein the remaining binding mixture comprises non-vesicular RNA;
(A) preparing a binding mixture comprising
   the remaining binding mixture comprising non-vesicular RNA,
   an acidic binding buffer comprising a buffering agent,
   wherein the pH of the binding buffer is ≤5.0, preferably ≤4.5 or ≤4.0,
   wherein the buffering agent comprises a carboxylic acid based buffer component, preferably selected from acetate and formate,
   (i) wherein the acidic binding buffer comprises the buffering agent in a concentration of 1M or less, such as 0.75M or less or 0.5M or less; or (ii) wherein the acidic binding buffer comprises the buffering agent in a concentration that lies in the range of 25 mM to 1000 mM, 50 mM to 750 mM, 100 mM to 650 mM or 200 mM to 500 mM,
   wherein the acidic binding buffer used in step (A) differs from the acidic binding buffer used in step (X) by one or more of the following features:
      (i) it has a lower pH;
      (ii) it has a higher ionic strength; and/or
      (iii) it comprises a crowding agent or comprises a higher concentration of a crowding agent compared to the acidic binding buffer used in step (X),
   and binding non-vesicular RNA to a solid phase comprising anion exchange groups, wherein the solid phase that is used in step (A) is provided by particles, preferably magnetic particles or a membrane,
   optionally wherein the anion exchange group of the solid phase comprises at least one amino group, wherein the amino group is part of an imidazole ring, preferably wherein the amino group is part of the imidazole ring of histamine,
   and optionally wherein the solid phase used in step (A) differs from the solid phase used in step (X) in that
   (i) it comprises more anion exchange groups, and/or
   (ii) it comprises stronger anion exchange groups;
(B) separating the solid phase with the bound non-vesicular RNA from the remaining binding mixture of (A);
(C) optionally washing the separated solid phase;
(D) recovering, preferably eluting, non-vesicular RNA from the solid phase, optionally wherein recovering in (D) comprises contacting the solid phase with a lysis buffer, optionally wherein the lysis buffer comprises guanidinium thiocyanate, and separating the solid phase from the eluate; and
(E) optionally purifying RNA from the eluate.
47. A kit for performing the method according to any one of items 1 to 46, comprising
   (a) an acidic binding buffer comprising a buffering agent;
   (b) a solid phase comprising anion exchange groups suitable for binding non-vesicular RNA under the conditions established by the acidic binding buffer (a);
   (c) optionally a wash solution; and
   (d) optionally a recovery solution.
48. The kit according to item 47, wherein the kit comprises
   (x) an acidic binding buffer comprising a buffering agent, wherein the acidic binding buffer (x) differs from the acidic binding buffer (a), wherein preferably, the acidic binding buffer (a) differs from the acidic binding buffer (x) by one or more of the following features
   (i) it has a lower pH;
   (ii) it has a higher ionic strength; and/or
   (iii) it comprises a crowding agent or comprises a higher concentration of a crowding agent compared to the acidic binding buffer used in step (X).
49. The kit according to item 47 or 48, wherein the kit comprises
   (b′) a second solid phase comprising anion exchange groups that differs from the solid phase (b), wherein preferably, the solid phase (b) differs from the solid phase (b′) in that (i) it comprises more anion exchange groups; and/or (ii) it comprises stronger anion exchange groups.
50. The kit according to item 48 and 49, wherein the kit comprises a binding buffer (x) and a second solid phase (b′) and wherein the second solid phase (b′) is suitable for binding extracellular vesicles under the conditions established by the acidic binding buffer (x).
51. The kit according to any one of items 47 to 50, wherein the kit has one or more of the following characteristics:
(i) the acidic binding buffer (a) has one or more of the characteristics as defined in any one of items 15 to 26,
(ii) the solid phase (b) has one or more of the characteristics as defined in any one of items 27 to 29, wherein preferably the solid phase is provided by magnetic particles; (iii) the wash buffer (c) has one or more of the characteristics as defined in any one of items 32 to 34; and/or
(iv) the recovery solution (d) is a lysis solution comprising guanidinium thiocyanate.
52. The kit according to any one of items 47 to 51, wherein the acidic binding buffer (x) has one or more of the characteristics as defined in any one of items 15 to 25.
53. The kit according to any one of items 47 to 52, wherein the solid phase (b′) has one or more of the characteristics as defined in any one of items 27 to 29, optionally wherein the solid phase (b) comprises anion exchange groups that comprise at least one quaternary amino group.
54. The kit according to any one of items 47 to 53, wherein the recovery solution (d) is an elution solution having one or more characteristics as defined in item 36.

This invention is not limited by the exemplary methods and materials disclosed herein, and any methods and materials similar or equivalent to those described herein can be used in the practice or testing of embodiments of this invention. Numeric ranges are inclusive of the numbers defining the range. The headings provided herein are not limitations of the various aspects or embodiments of this invention which can be read by reference to the specification as a whole.

As used in the subject specification, items and claims, the singular forms “a”, “an” and “the” include plural aspects unless the context clearly dictates otherwise. The terms “include,” “have,” “comprise” and their variants are used synonymously and are to be construed as non-limiting. Further components and steps may be present. Throughout the specification, where compositions are described as comprising components or materials, it is additionally contemplated that the compositions can in embodiments also consist essentially of, or consist of, any combination of the recited components or materials, unless described otherwise. Reference to “the disclosure” and “the invention” and the like includes single or multiple aspects taught herein; and so forth. Aspects taught herein are encompassed by the term “invention”.

It is preferred to select and combine preferred embodiments described herein and the specific subject-matter arising from a respective combination of preferred embodiments also belongs to the present disclosure.

### EXAMPLES

It should be understood that the following examples are for illustrative purpose only and are not to be construed as limiting this invention in any manner.

The following examples demonstrate the important advantages of the method of the present invention in effectively enriching non-vesicular RNA, such as non-vesicular miRNA, from the extracellular fraction of a body fluid, such as plasma. The examples show that the teachings of the present invention allow to improve the binding of non-vesicular RNA, such as non-vesicular miRNA, to a solid phase comprising anion exchange groups. In particular, the binding of non-vesicular RNA, such as non-vesicular miRNA, can be improved by (i) a more acid pH (e.g. in the range of approx. 2.5 to 5.0, such as 3.0 to 4.5 or 3.0 to 4.0), (ii) an increase of the ionic strength, (iii) the use of a solid phase that comprises a high number/density of anion exchange groups, (iv) the addition of a crowding agent, such as polyethylene glycol or (v) a combination of two or more of (i) to (iv).

As is furthermore demonstrated by the below examples, when using one or more of these measures to improve binding of non-vesicular RNA, such as non-vesicular miRNA, to the anion exchange solid phase, binding of extracellular vesicles to said solid phase is not or only moderately affected. As shown in the examples, this allows improving the binding and thus recovery of non-vesicular RNA, such as non-vesicular miRNA, in a method that recovers from the same binding mixture extracellular vesicles by binding to the anion exchange solid phase. The bound extracellular vesicles and bound non-vesicular RNA can then be further processed in order to purify an RNA fraction that comprises vesicular RNA and non-vesicular RNA (such as non-vesicular miRNA). Furthermore, the identified differences in the binding behavior of non-vesicular RNA and extracellular vesicles can be used to enrich extracellular vesicles (and thus vesicular RNA) and non-vesicular RNA (such as non-vesicular miRNA) in separate fractions. The below examples illustrate suitable embodiments wherein extracellular vesicles and non-vesicular RNAs are bound in separate binding steps. This allows to provide vesicular RNA and non-vesicular RNA as separate fractions.

### Abbreviations

EV extracellular vesicles
GTC guanidinium thiocyanate
miRNA microRNA
PCR polymerase chain reaction
PEG polyethylene glycol
RT-PCR reverse transcription polymerase chain reaction

The exoRNeasy Midi/Maxi Kit (QIAGEN, Hilden) is commercially available. In brief, after separation of the solid phase with the bound RNA, a phenol/guanidinium thiocyanate-based lysis buffer (e.g. QIAzol Lysis Reagent, QIAGEN, Hilden) is added to the spin column. Afterwards, the spin column is centrifuged and subsequently the flow-through/lysate is collected into a new tube. Briefly the tube containing the lysate is vortexed and incubated at room temperature for 5 min. This step promotes dissociation of nucleoprotein complexes. Next, chloroform is added to the lysate and the sample is agitated, e.g. vortexed for 15 s. Subsequently, the sample is incubated for about 2 min to 5 min and then centrifuged to remove the phenol. After centrifugation, the sample separates into three phases: an upper, colorless aqueous phase containing RNA, a thin, white interphase; and a lower, red organic phase. The aqueous phase comprising the RNA is transferred to a new tube and ethanol is added. The sample is transferred to a new spin column (e.g. RNeasy spin column, QIAGEN, Hilden) to bind the RNA. After several washing steps, the RNA can be eluted with RNase-free water. The solid phase used for binding in said kit may also be used in the present invention.

### Example 1: Influence of pH and Ionic Strength

1 ml human plasma from a healthy donor was mixed with an equal volume of different binding buffers (see Table 1 below) and incubated with 0.5 mg magnetic anion exchange beads carrying histamine functional groups for 10 min on an end-over-end shaker. The beads were collected on a magnetic rack, the supernatant was removed and the beads were washed once in 235 mM acetate pH5. The beads were then treated with 700 μl QIAzol lysis reagent (pH adjusted to 8 using a 2M GTC, 2M Trizma base solution) to lyse the bound vesicles and elute their contents. The lysis step also dissolved bound nucleoprotein complexes and their constituents were then eluted.

RNA was isolated from the QIAzol eluates following the miRNeasy Micro procedure, using 90 μl chloroform in the phase separation step.

For reference, an additional 1 ml of the same plasma sample was processed using the exoRNeasy Maxi Kit.

Relative quantitation of representative miRNAs let-7a (vesicular) and miR-122 (non-vesicular) was done by quantitative real-time RT-PCR using the miRCURY LNA RT and SYBR Green PCR kits, with the respective miRCURY assays.

All isolations were performed in duplicates, for each eluate two replicate assays were performed.

TABLE 1
Binding buffers.

| Binding buffer | pH | Concentration |
|---|---|---|
| Formate | 3 | 465 mM |
| Formate | 3 | 240 mM |
| Formate | 3 | 100 mM |
| Formate | 3.5 | 465 mM |
| Formate | 3.5 | 240 mM |
| Formate | 3.5 | 100 mM |
| Acetate | 3.5 | 465 mM |
| Acetate | 3.5 | 240 mM |
| Acetate | 3.5 | 100 mM |
| Acetate | 4 | 465 mM |
| Acetate | 4 | 240 mM |
| Acetate | 4 | 100 mM |

### Results:

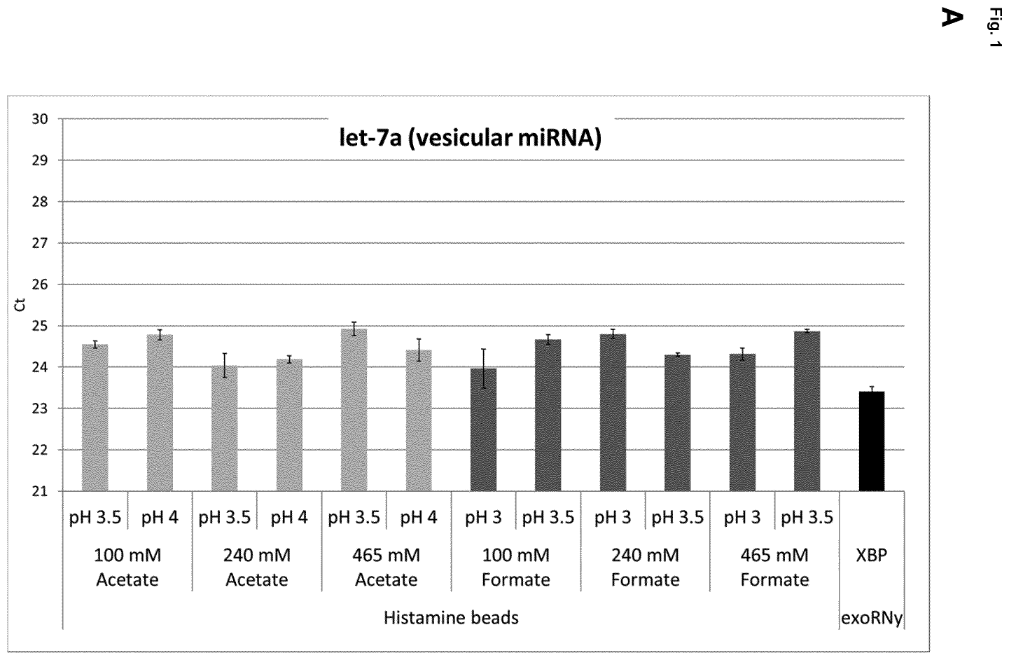

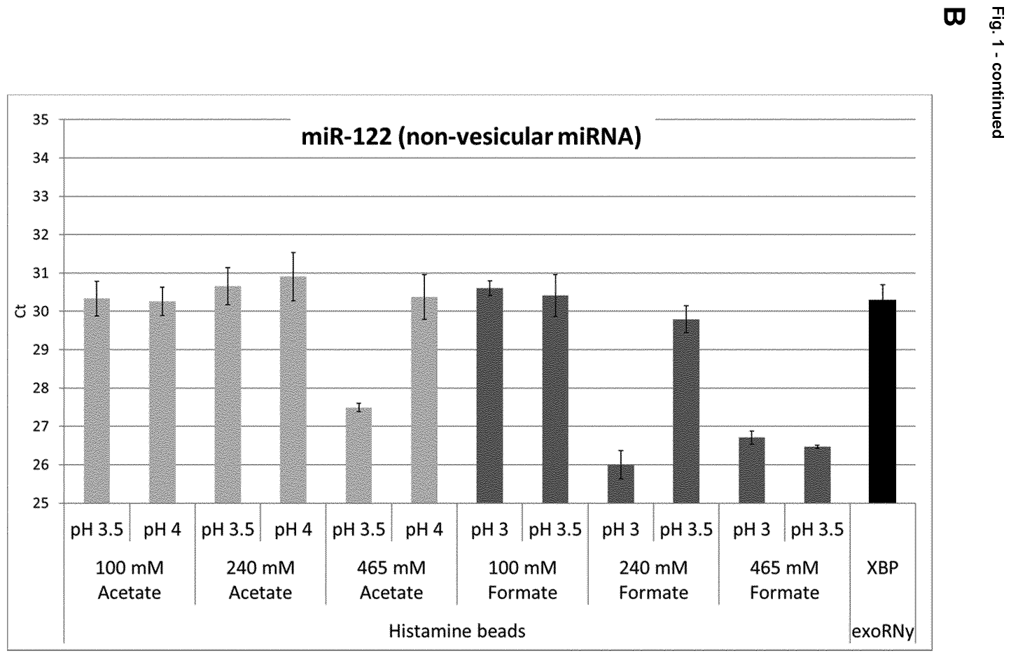

While Ct values for the vesicular miRNA let-7a all vary about 1 cycle (FIG. 1A), Ct values for non-vesicular miR-122 are 3-4 cycles lower in conditions with low pH and higher ionic strength of the buffers, i.e. the binding buffers with a pH of 3.5 containing 465 mM acetate or formate, respectively, and the binding buffers with a pH of 3 containing 240 mM formate or 465 mM formate, respectively, corresponding to an approximately 8-16-fold higher recovery (FIG. 1B).

### Example 2: Influence of Ionic Strength

1 ml human plasma from a healthy donor was processed according to the exoRNeasy Maxi protocol, with the following modifications: QIAzol was adjusted to pH8 as in Example 1, binding buffer XBP was replaced by 50, 100, 240, or 465 mM acetate buffer, wash buffer XWP was replaced by 235 mM acetate pH5. The unmodified exoRNeasy protocol was included as reference. All samples were processed in duplicates.

Relative quantitation of representative miRNAs miR-150 (vesicular) and miR-122 (non-vesicular) was done by quantitative real-time RT-PCR using the miRCURY LNA RT and SYBR Green PCR kits, with the respective miRCURY assays.

### Results

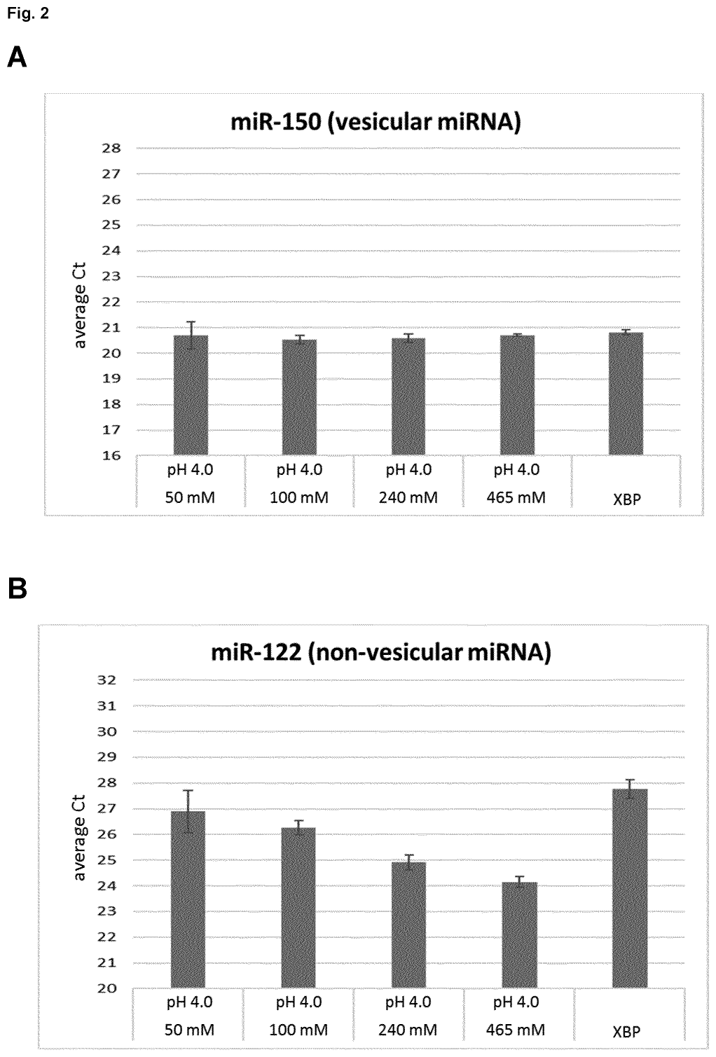

While Ct values for the vesicular miRNA miR-150 are essentially unchanged between all conditions (FIG. 2A), Ct values of the non-vesicular miRNA miR-122 improve gradually with increasing ionic strength of the buffer (FIG. 2B).

### Example 3: Influence of Binding Matrix

1 ml human plasma from a healthy donor was mixed with an equal volume of 465 mM acetate pH4 and incubated with 0.5 mg magnetic anion exchange beads carrying histamine or His10 peptide as functional groups for 10 min on an end-over-end shaker. The beads were collected on a magnetic rack, the supernatant was removed and the beads were washed once in 235 mM acetate pH5. The beads were then further processed and RNA isolated as in Example 1.

For reference, an additional 1 ml of the same plasma was processed using the exoRNeasy Maxi Kit according to the unmodified exoRNeasy protocol.

Relative quantitation of representative miRNAs let-7a (vesicular) and miR-122 (non-vesicular) was done by quantitative real-time RT-PCR using the miRCURY LNA RT and SYBR Green PCR kits, with the respective miRCURY assays.

All isolations were performed in duplicates, for each eluate two replicate assays were performed.

### Results

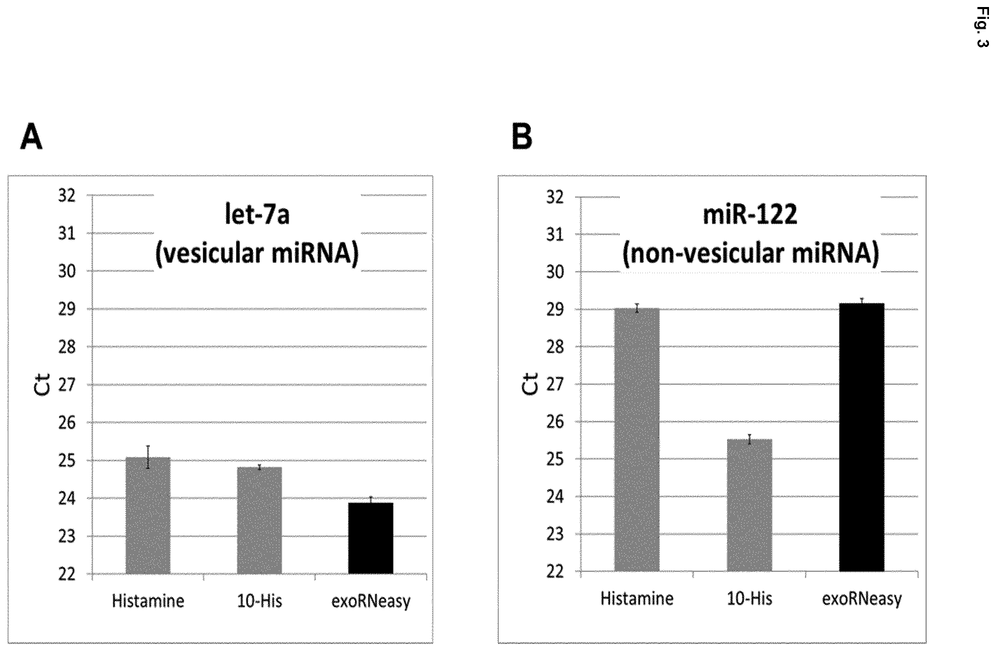

Again, Ct values and, accordingly, recovery of vesicular let-7a were not significantly different between the two bead types (FIG. 3A). For non-vesicular miR-122, Ct values increased by 3-4 cycles due to the higher charge density of the His₁₀ beads (FIG. 3B).

### Example 4: Influence of Molecular Crowding (PEG)

1 ml human plasma from a healthy donor was mixed with an equal volume of 465 mM acetate pH4 with or without 5% PEG 8000 and incubated with 0.5 mg magnetic anion exchange beads carrying histamine functional groups for 10 min on an end-over-end shaker. The beads were collected on a magnetic rack, the supernatant was removed and the beads were washed once in 235 mM acetate pH5. The beads were then further processed and RNA isolated as in Example 1.

For reference, an additional 1 ml of the same plasma was processed using the exoRNeasy Maxi Kit according to the unmodified exoRNeasy protocol.

Relative quantitation of representative miRNAs miR-150 (vesicular) and miR-122 (non-vesicular) was done by quantitative real-time RT-PCR using the miRCURY LNA RT and SYBR Green PCR kits, with the respective miRCURY assays.

All isolations were performed in duplicates, for each eluate two replicate assays were performed.

### Results

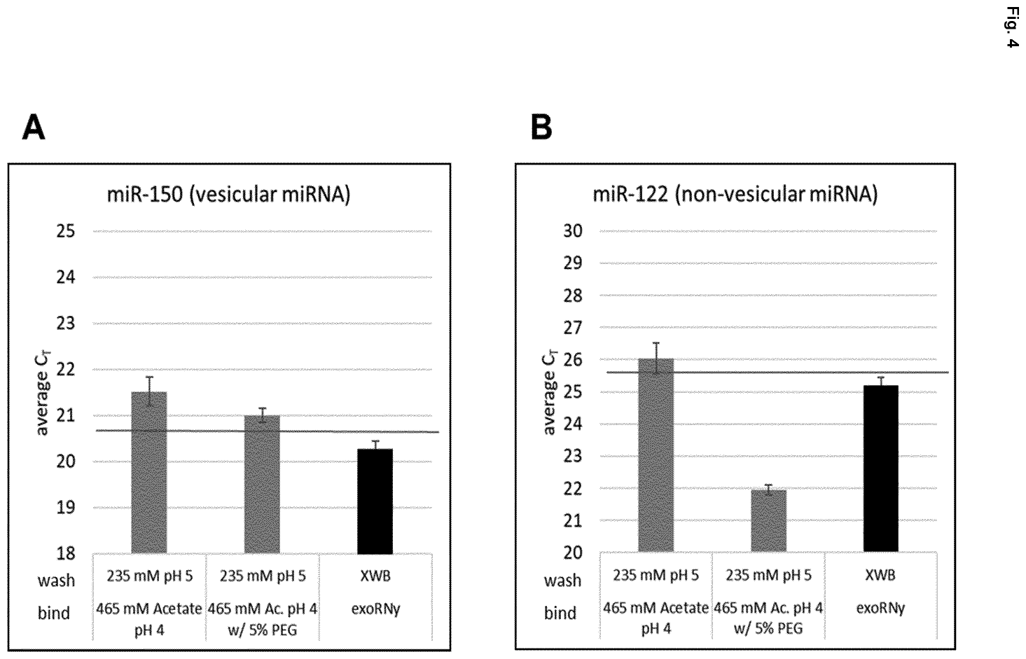

Adding PEG to the binding buffer changed recovery of vesicular miR-150 only slightly (FIG. 4A), whereas recovery of non-vesicular miR-122 improved by 5 cycles, or about 30-fold (FIG. 4B).

### Example 5: Two-Step Procedure

1 ml human plasma from a healthy donor was mixed with an equal volume of 465 mM acetate pH4 and incubated with 0.5 mg magnetic anion exchange beads carrying histamine functional groups for 10 min on an end-over-end shaker. The beads were collected on a magnetic rack, the supernatant retained for extraction of unbound material, and the beads washed once in 235 mM acetate pH5. The beads were then further processed and RNA isolated as in Example 1.

For reference, an additional 1 ml of the same plasma was processed using the exoRNeasy Maxi Kit according to the unmodified exoRNeasy protocol. The flow-through of the exoEasy spin column was retained for extraction of unbound material.

Supernatant and flow-through fractions were applied to fresh exoEasy Maxi spin columns and the columns further processed according to the exoRNeasy Maxi protocol.

Relative quantitation of representative miRNAs miR-150 and miR-122 was done by quantitative real-time RT-PCR using the miRCURY LNA RT and SYBR Green PCR kits, with the respective miRCURY assays.

All isolations were performed in duplicates, for each eluate two replicate assays were performed.

### Results

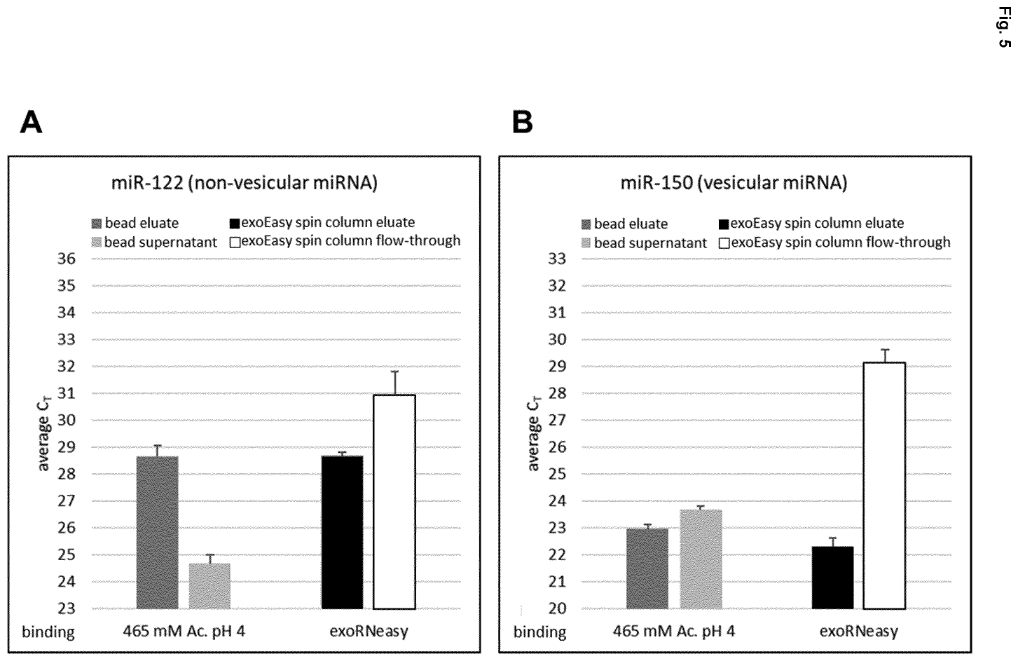

The results show efficient recovery of non-vesicular miR-122 (second left bar of FIG. 5A) from the supernatant of the EV binding to magnetic beads which bound extracellular vesicles comprising the vesicular miRNA-150 (left bar of FIG. 5B). In contrast, only a low amount of non-vesicular miR-122 bound to the magnetic beads (left bar of FIG. 5A) and could be recovered from the flow-through of the reference method (right bar of FIG. 5A).

Membranes in the exoEasy spin columns contain quaternary amino groups at high density and therefore have a higher affinity to both EVs and nucleic acids.

1-16. (canceled)

17. A method for enriching non-vesicular RNA from a cell-free or cell-depleted biological sample that includes non-vesicular RNA, the method comprising:
preparing a binding mixture comprising
the biological sample, and
an acidic binding buffer comprising a buffering agent;
binding non-vesicular RNA in the binding mixture to a solid phase comprising anion exchange groups; and
separating the solid phase with the bound non-vesicular RNA from the binding mixture.

18. The method according to claim 17, wherein the non-vesicular RNA in the biological sample comprises non-vesicular miRNA.

19. The method according to claim 17, wherein the biological sample also includes extracellular vesicles.

20. The method according to claim 17, wherein the method further comprises washing the separated solid phase.

21. The method according to claim 17, wherein the method further comprises recovering the non-vesicular RNA from the solid phase.

22. The method according to claim 21, wherein the non-vesicular RNA is recovered from the solid phase by eluting.

23. The method according to claim 22, further comprising purifying the non-vesicular RNA from the elute.

24. The method according to claim 19, wherein the method comprises binding the non-vesicular RNA and the extracellular vesicles to the solid phase, and wherein the method comprises separating the solid phase with the bound non-vesicular RNA and the bound extracellular vesicles from the binding mixture.

25. The method according to claim 24, wherein the method further comprises recovering either
the non-vesicular RNA and the extracellular vesicles, or
the non-vesicular RNA and extracted vesicular RNA from the solid phase.

26. The method according to claim 17, wherein the cell-free or cell-depleted biological sample is depleted of extracellular vesicles.

27. A method for sequentially enriching extracellular vesicles and non-vesicular RNA from a cell-free or cell-depleted biological sample that includes extracellular vesicles and non-vesicular RNA, the method comprising:
preparing a first binding mixture comprising
the biological sample, and
an acidic binding buffer comprising a buffering agent;
binding extracellular vesicles in the first binding mixture to a first solid phase comprising anion exchange groups;
separating the first solid phase with the bound extracellular vesicles from what remains of the first binding mixture, wherein what remains of the first binding mixture comprises non-vesicular RNA;
preparing a second binding mixture comprising what remains of the first binding mixture;
binding non-vesicular RNA in the second binding mixture to a second solid phase comprising anion exchange groups; and
separating the second solid phase with the bound non-vesicular RNA from what remains of the first binding mixture.

28. The method according to claim 27, wherein the non-vesicular RNA in the biological sample comprises non-vesicular miRNA.

29. The method according to claim 27, wherein the second binding mixture further comprises an acidic binding buffer comprising a buffering agent.

30. The method according to claim 27, wherein the method further comprises washing the second separated solid phase.

31. The method according to claim 27, wherein the method further comprises recovering the non-vesicular RNA from the second solid phase.

32. The method according to claim 31, wherein the non-vesicular RNA is recovered from the second solid phase by eluting.

33. The method according to claim 32, further comprising purifying the non-vesicular RNA from the elute.

34. The method according to claim 27, wherein the second solid phase is different than the first solid phase, and wherein the second solid phase comprises
(i) more anion exchange groups than the first solid phase; and/or
(ii) stronger anion exchange groups than the first solid phase.

35. The method according to claim 34, wherein the chemical compositions of the first binding mixture and the second binding mixture are the same, and wherein binding of the non-vesicular RNA is achieved because the second solid phase is different than the first solid phase.

36. The method according to claim 27, wherein preparing the second binding mixture comprises modifying what remains of the first binding mixture to achieve binding of non-vesicular RNA to the second solid phase, wherein modifying includes one or more of
(i) lowering the pH,
(ii) increasing the ionic strength, and/or
(iii) adding a crowding agent.

37. The method according to claim 36, wherein the crowding agent is polyethylene glycol.

38. The method according to claim 27, wherein the second binding mixture is contacted with an acidic binding buffer comprising a different buffering agent than the acidic binding buffer in the first binding mixture, wherein the acidic binding buffer in the second binding mixture differs from the acidic binding buffer in the first binding mixture by one or more of the following features:
(i) it has a lower pH;
(ii) it has a higher ionic strength; and/or
(iii) it comprises a crowding agent or comprises a higher concentration of a crowding agent than in the first binding mixture.

39. The method according to claim 17, wherein the pH of the acidic binding buffer is ≤5.0.

40. The method according to claim 17, wherein the pH of the acidic binding buffer is in the range of 2.5 to 5.0.

41. The method according to claim 38, wherein the pH of the acidic binding buffer in one or both of the first binding mixture and the second binding mixture is ≤5.0.

42. The method according to claim 38, wherein the pH of the acidic binding buffer in one or both of the first binding mixture and the second binding mixture is in the range of 2.5 to 5.0.

43. The method according to claim 17, wherein the method has one or more of the following features:
(i) the acidic binding buffer comprises a carboxylic acid based buffering agent;
(ii) the acidic binding buffer comprises the buffering agent in a concentration of 1M or less;
(iii) the acidic binding buffer comprises a non-buffering salt;
(iv) the acidic binding buffer comprises a carboxylic acid based buffering agent that comprises a component selected from citrate, oxalate, formate, acetate, propionate, lactate and tartrate;
(v) the acidic binding buffer comprises the buffering agent in a concentration in the range of 25 mM to 1000 mM;
(vi) the acidic binding buffer comprises a crowding agent;
(vii) the acidic binding buffer comprises a crowding agent selected from a poly(alkylene oxide) polymer and dextran;
(viii) the acidic binding buffer comprises polyethylene glycol as a crowding agent;
(ix) the solid phase is provided by particles or a porous membrane or filter;
(x) the solid phase comprises anion exchange groups that comprise at least one primary, secondary, tertiary or quaternary amino group; and
(xi) the solid phase is provided by particles, and wherein the particles are magnetic particles.

44. The method according to claim 27, wherein the method has one or more of the following features:
(i) the pH of the acidic binding buffer is ≤5.0;
(ii) the second binding mixture further comprises an acidic binding buffer and the pH of the acidic binding buffer in one or both of the first binding mixture and the second binding mixture is ≤5.0;
(iii) the pH of the acidic binding buffer is in the range of 2.5 to 5.0;
(iv) the second binding mixture further comprises an acidic binding buffer and the pH of the acidic binding buffer in one or both of the first binding mixture and the second binding mixture is in the range of 2.5 to 5.0;
(v) the acidic binding buffer comprises a carboxylic acid based buffering agent;
(vi) the acidic binding buffer comprises the buffering agent in a concentration of 1M or less;
(vii) the acidic binding buffer comprises a non-buffering salt;
(viii) the acidic binding buffer comprises a carboxylic acid based buffering agent that comprises a component selected from citrate, oxalate, formate, acetate, propionate, lactate and tartrate;
(ix) the acidic binding buffer comprises the buffering agent in a concentration in the range of 25 mM to 1000 mM;
(xi) the acidic binding buffer comprises a crowding agent;
(xi) the acidic binding buffer comprises a crowding agent selected from a poly(alkylene oxide) polymer and dextran;
(xii) the acidic binding buffer comprises polyethylene glycol as a crowding agent;
(xiii) the solid phase is provided by particles or a porous membrane or filter;
(xiv) the solid phase comprises anion exchange groups that comprise at least one primary, secondary, tertiary or quaternary amino group; and
(xv) the solid phase is provided by particles, and wherein the particles are magnetic particles.

45. The method according to claim 38, wherein the method has one or more of the following features:
(i) the acidic binding buffer comprises a carboxylic acid based buffering agent;
(ii) the acidic binding buffer comprises the buffering agent in a concentration of 1M or less;
(iii) the acidic binding buffer comprises a non-buffering salt;
(iv) the acidic binding buffer comprises a carboxylic acid based buffering agent that comprises a component selected from citrate, oxalate, formate, acetate, propionate, lactate and tartrate;
(v) the acidic binding buffer comprises the buffering agent in a concentration in the range of 25 mM to 1000 mM;
(vi) the acidic binding buffer comprises a crowding agent;
(vii) the acidic binding buffer comprises a crowding agent selected from a poly(alkylene oxide) polymer and dextran;
(viii) the acidic binding buffer comprises polyethylene glycol as a crowding agent;
(ix) the solid phase is provided by particles or a porous membrane or filter;
(x) the solid phase comprises anion exchange groups that comprise at least one primary, secondary, tertiary or quaternary amino group; and
(xi) the solid phase is provided by particles, and wherein the particles are magnetic particles.

46. The method according to claim 38, wherein the non-vesicular RNA is recovered from the second solid phase by eluting.

47. The method according to claim 46, further comprising purifying the non-vesicular RNA from the elute.

48. The method according to claim 17, wherein the non-vesicular RNA to be enriched is non-vesicular miRNA, wherein the biological sample is selected from plasma and serum,
wherein the acidic binding buffer has a pH≤5.0,
wherein the buffering agent comprises a carboxylic acid based buffer component,
wherein the acidic binding buffer comprises the buffering agent in a concentration that lies in the range of 25 mM to 1000 mM, wherein the solid phase comprises anion exchange groups, and
wherein the solid phase is provided by particles.

49. The method according to claim 27, wherein the non-vesicular RNA to be enriched is non-vesicular miRNA and the extracellular vesicles comprise vesicular miRNA, wherein the biological sample in the first binding mixture is selected from plasma or serum, and wherein the acidic binding buffer of the second binding mixture is ≤5.0.

50. The method according to claim 38, wherein the non-vesicular RNA to be enriched is non-vesicular miRNA and the extracellular vesicles comprise vesicular miRNA, wherein the biological sample in the first binding mixture is selected from plasma or serum, and wherein the acidic binding buffer of the second binding mixture is ≤5.0.

51. A kit for performing the method according to claim 17, the kit comprising:
(a) an acidic binding buffer comprising a buffering agent; and
(b) a solid phase comprising anion exchange groups suitable for binding non-vesicular RNA under conditions established by the acidic binding buffer (a).

52. The kit according to claim 51, further comprising a wash solution.

53. The kit according to claim 51, further comprising a recovery solution.

54. The kit according to claim 51, wherein the kit further comprises one or more of the following components:
(x) an additional acidic binding buffer comprising an additional buffering agent, wherein the additional acidic binding buffer (x) differs from the acidic binding buffer (a); and
(b′) a second solid phase (b′) comprising anion exchange groups that differs from the solid phase (b).

55. The kit according to claim 54, wherein the acidic binding buffer (a) differs from the acidic binding buffer (x) by one or more of the following features
(i) it has a lower pH;
(ii) it has a higher ionic strength; and/or
(iii) it comprises a crowding agent or comprises a higher concentration of a crowding agent compared to the acidic binding buffer used in step (X).

56. The kit according to claim 54, wherein the solid phase (b) differs from the second solid phase (b′) in that
(i) it comprises more anion exchange groups; and/or
(ii) it comprises stronger anion exchange groups.

57. The kit according to claim 56, wherein the second solid phase (b′) is suitable for binding extracellular vesicles under conditions established by the acidic binding buffer (x).
